(19) United States
(12) Patent Application Publication
(10) Pub. No.: US 2026/0199017 A1
(43) Pub. Date: Jul. 16, 2026

(54) METHOD OF AND SYSTEM FOR GENERATING A PREDICTION OF AN IMPACT OF AN INTERVENTION ON 3D KINEMATIC MOTION OF A JOINT BEFORE OR DURING AN OPERATION

(71) Applicant: EMOVI INC., Montréal (CA)

(72) Inventors: Alexandre FUENTES, Montréal (CA); Alix Franco Maurice CAGNIN, Lyon (FR); Eric SZMUTNY, Saint-Armand (CA); Nicola HAGEMEISTER, Montréal (CA); Bianca MAROIS, Montréal (CA); Neila MEZGHANI, Montréal (CA); Philippe LANDRY, Montréal (CA)

(73) Assignee: EMOVI INC., Montréal (CA)

(21) Appl. No.: 19/296,659

(22) Filed: Aug. 11, 2025

Related U.S. Application Data
(60) Provisional application No. 63/745,628, filed on Jan. 15, 2025.

(51) Int. Cl. A61B 34/10; A61B 90/00

(52) U.S. Cl. CPC A61B 34/10; A61B 90/361; A61B 2034/101

(57) ABSTRACT

There is provided a method and system for generating a prediction of an impact of an intervention on three-dimensional (3D) kinematic motion of a joint before or during an operation. The method includes: receiving 3D kinematic motion data; receiving complementary surgical data; merging the 3D kinematic motion data and the complementary surgical data, thereby creating a merged data set; deriving, from the merged data set, functional parameters characterizing the 3D kinematic motion; determining an initial prediction of the 3D kinematic motion before or during the operation based on the functional parameters; generating a refined prediction of the 3D kinematic motion based on the initial prediction and a selection of surgical features; and generating a graphical output before or during the operation to be output to a display, the graphical output including a graphical representation based on the initial prediction or the refined prediction.

### CROSS-REFERENCE TO RELATED APPLICATIONS

This application claims the benefit of U.S. Provisional Patent Application No. 63/745,628, filed on Jan. 15, 2025, which is incorporated by reference herein in its entirety.

### FIELD OF THE INVENTION

The present technology relates to tools for visualization of training, planning surgery, and/or surgical procedures and more specifically to methods, systems, and non-transitory storage mediums for generating predictions of the impact of an intervention on kinematic motion of a joint before or during an operation.

### BACKGROUND OF THE INVENTION

Systems dealing with surgical training, surgery planning, and intra-operative visualization are mostly based on information obtained by reviewing medical imagery in static conditions and/or 3D simulation based on static information or dynamic simulated information. These systems include radiography, magnetic resonance, CT scans (e.g., non-weight-bearing and weight-bearing), KT-1000 arthrometer, specified clinical tests (e.g., pivot shift test and Lachman test) and the like. Such exams however remain limited in terms of their capacity to evaluate various functional aspects of the different joints, and typically cannot be performed while the joint is moving (i.e., they are static in nature).

Other existing methods quantify and display real-time passive behavior of joint movement during surgery, while surgical staff move the joint in different directions. Since these procedures rely on manual testing, they are tainted by a certain amount of subjectivity as data is greatly influenced by the position and maneuvering of the clinical staff and the fact that these movements are passive (i.e., not using patient muscles to actively move the joint) and performed during non-weight-bearing tasks (e.g., open-chain kinematics). Conventional systems address one aspect of this issue by using tensioners to apply consistent stress forces within the joint throughout passive movements of the joint. While it allows more standardization from one patient to another, the most appropriate amount of stress to apply (e.g., in each compartment of the joint, if applicable) for each patient is still unknown and, furthermore, it is not representative of what the joint could undergo in weight-bearing conditions.

There exists a need for an improved system and/or method that overcomes the deficiencies described above.

### SUMMARY OF THE INVENTION

It is an object of the present technology to ameliorate at least some of the inconveniences present in the prior art. One or more implementations of the present technology may provide and/or broaden the scope of approaches to and/or methods of achieving the aims and objects of the present technology.

According to one aspect of the present technology, there is provided a system for generating a prediction of an impact of an intervention on three-dimensional (3D) kinematic motion of a joint before or during an operation. The system comprises a display and at least one processor. The display is configured to display a representation of the prediction of the impact of the intervention on the 3D kinematic motion of the joint. The at least one processor is operatively connected to the display and a non-transitory storage medium having stored thereon computer-readable instructions. The computer-readable instructions, when executed by the at least one processor, cause the at least one processor to receive 3D kinematic motion data of the joint, the 3D kinematic motion data relating to the 3D kinematic motion during at least one of a weight-bearing action or the weight-bearing action in combination with motion during a non-weight-bearing action; receive complementary surgical data that relate to the joint or the operation; merge the 3D kinematic motion data and the complementary surgical data, thereby creating a merged data set; derive, from the merged data set, one or more functional parameters characterizing the 3D kinematic motion; determine an initial prediction of the 3D kinematic motion before or during the operation based on the one or more functional parameters; generate a refined prediction of the 3D kinematic motion based on the initial prediction and a selection of one or more surgical features that relate to the operation; and generate a graphical output before or during the operation to be output to the display, the graphical output comprising a graphical representation based on at least one of the initial prediction or the refined prediction.

According to another aspect of the present technology, there is provided a method for generating a prediction of an impact of an intervention on three-dimensional (3D) kinematic motion of a joint before or during an operation. The method comprises: receiving 3D kinematic motion data of the joint, the 3D kinematic motion data relating to the 3D kinematic motion during at least one of a weight-bearing action or the weight-bearing action in combination with motion during a non-weight-bearing action; receiving complementary surgical data that relate to the joint or the operation; merging the 3D kinematic motion data and the complementary surgical data, thereby creating a merged data set; deriving, from the merged data set, one or more functional parameters characterizing the 3D kinematic motion; determining an initial prediction of the 3D kinematic motion before or during the operation based on the one or more functional parameters; generating a refined prediction of the 3D kinematic motion based on the initial prediction and a selection of one or more surgical features that relate to the operation; and generating a graphical output before or during the operation to be output to the display, the graphical output comprising a graphical representation based on at least one of the initial prediction or the refined prediction.

Implementations of the present technology each have at least one of the above-mentioned objects and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects, and advantages of implementations of the present technology will become apparent from the following description and the accompanying drawings.

### Terms and Definitions

In the context of the present specification, a “server” is a computer program that is running on appropriate hardware and is capable of receiving requests (e.g., from computing devices) over a network (e.g., a communication network), and carrying out those requests, or causing those requests to be carried out. The hardware may be one physical computer or one physical computer system, but neither is required to be the case with respect to the present technology. In the present context, the use of the expression a “server” is not intended to mean that every task (e.g., received instructions or requests) or any particular task will have been received, carried out, or caused to be carried out, by the same server (i.e., the same software and/or hardware); it is intended to mean that any number of software elements or hardware devices may be involved in receiving/sending, carrying out or causing to be carried out any task or request, or the consequences of any task or request; and all of this software and hardware may be one server or multiple servers, both of which are included within the expressions “at least one server” and “a server”.

In the context of the present specification, “computing device” is any computing apparatus or computer hardware that is capable of running software appropriate to the relevant task at hand. Thus, some (non-limiting) examples of computing devices include general purpose personal computers (desktops, laptops, netbooks, etc.), mobile computing devices, smartphones, and tablets, and network equipment such as routers, switches, and gateways. It should be noted that a computing device in the present context is not precluded from acting as a server to other computing devices. The use of the expression “a computing device” does not preclude multiple computing devices being used in receiving/sending, carrying out or causing to be carried out any task or request, or the consequences of any task or request, or steps of any method described herein. In the context of the present specification, a “client device” refers to any of a range of end-user client computing devices, associated with a user, such as personal computers, tablets, smartphones, and the like.

In the context of the present specification, the expression “computer-readable storage medium” (also referred to as “storage medium” and “storage”) is intended to include non-transitory media of any nature and kind whatsoever, including without limitation RAM, ROM, disks (CD-ROMs, DVDs, floppy disks, hard drives, etc.), USB keys, solid state drives, tape drives, etc. A plurality of components may be combined to form the computer information storage media, including two or more media components of a same type and/or two or more media components of different types.

In the context of the present specification, a “database” is any structured collection of data, irrespective of its particular structure, the database management software, or the computer hardware on which the data is stored, implemented or otherwise rendered available for use. A database may reside on the same hardware as the process that stores or makes use of the information stored in the database or it may reside on separate hardware, such as a dedicated server or plurality of servers.

In the context of the present specification, the expression “information” includes information of any nature or kind whatsoever capable of being stored in a database. Thus, information includes, but is not limited to, audiovisual works (images, movies, sound records, presentations etc.), data (location data, numerical data, non-numerical data, metadata, categorical data, etc.), text (opinions, comments, questions, messages, etc.), documents, spreadsheets, lists of words, etc.

In the context of the present specification, unless expressly provided otherwise, an “indication” of an information element may be the information element itself or a pointer, reference, link, or other indirect mechanism enabling the recipient of the indication to locate a network, memory, database, or other computer-readable medium location from which the information element may be retrieved. For example, an indication of a document could include the document itself (i.e., its contents), or it could be a unique document descriptor identifying a file with respect to a particular file system, or some other means of directing the recipient of the indication to a network location, memory address, database table, or other location where the file may be accessed. As one skilled in the art would recognize, the degree of precision required in such an indication depends on the extent of any prior understanding about the interpretation to be given to information being exchanged as between the sender and the recipient of the indication. For example, if it is understood prior to a communication between a sender and a recipient that an indication of an information element will take the form of a database key for an entry in a particular table of a predetermined database containing the information element, then the sending of the database key is all that is required to effectively convey the information element to the recipient, even though the information element itself was not transmitted as between the sender and the recipient of the indication.

In the context of the present specification, the expression “communication network” is intended to include a telecommunications network such as a computer network, the Internet, a telephone network, a Telex network, a TCP/IP data network (e.g., a WAN network, a LAN network, etc.), and the like. The term “communication network” includes a wired network or direct wired connection, and wireless media such as acoustic, radio frequency (RF), infrared, and other wireless media, as well as combinations of any of the above.

In the context of the present specification, the words “first”, “second”, “third”, etc. have been used as adjectives only for the purpose of allowing for distinction between the nouns that they modify from one another, and not for the purpose of describing any particular relationship between those nouns. Thus, for example, it should be understood that, the use of the terms “server” and “third server” is not intended to imply any particular order, type, chronology, hierarchy, or ranking (for example) of/between the servers, nor is their use (by itself) intended to imply that any “second server” must necessarily exist in any given situation. Further, as is discussed herein in other contexts, reference to a “first” element and a “second” element does not preclude the two elements from being the same actual real-world element. Thus, for example, in some instances, a “first” server and a “second” server may be the same software and/or hardware; in other cases they may be different software and/or hardware.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 depicts a schematic diagram of an electronic device in accordance with one or more non-limiting implementations of the present technology.

FIG. 2 depicts a schematic diagram of a communication system in accordance with one or more non-limiting implementations of the present technology.

FIG. 3 depicts a flowchart of a method for generating a prediction of 3D kinematic motion of a joint before or during an operation in accordance with one or more non-limiting implementations of the present technology.

FIG. 4 depicts a flowchart of an initial prediction algorithm in accordance with one or more non-limiting implementations of the present technology.

FIG. 5 depicts a flowchart of an iterative algorithm in accordance with one or more non-limiting implementations of the present technology.

FIG. 6 depicts a flowchart of a boundaries algorithm in accordance with one or more non-limiting implementations of the present technology.

FIG. 7 depicts a flowchart of a post-op algorithm in accordance with one or more non-limiting implementations of the present technology.

FIG. 8 depicts a flowchart of a prioritization algorithm in accordance with one or more non-limiting implementations of the present technology.

FIG. 9 depicts a flowchart of a method for generating and displaying a prediction of 3D kinematic motion of a joint during weight-bearing activity before or during an operation in accordance with one or more non-limiting implementations of the present technology.

FIG. 10 depicts a schematic diagram of a user interface for an initial prediction of 3D kinematic motion of a joint in accordance with one or more non-limiting implementations of the present technology.

FIG. 11 depicts a schematic diagram of a user interface for a refined prediction of 3D kinematic motion of a joint in accordance with one or more non-limiting implementations of the present technology.

FIG. 12 depicts a schematic diagram of a user interface for a comparison of an initial prediction with a refined prediction of 3D kinematic motion of a joint in accordance with one or more non-limiting implementations of the present technology.

FIG. 13 depicts a schematic diagram of a user interface for a comparison of an initial prediction with an additional (or subsequent) prediction of 3D kinematic motion of a joint in accordance with one or more non-limiting implementations of the present technology.

FIG. 14 depicts a schematic diagram of a user interface for a comparison of a refined prediction with an additional (or subsequent) prediction of 3D kinematic motion of a joint in accordance with one or more non-limiting implementations of the present technology.

FIG. 15 depicts a schematic diagram of a user interface for iterative modification of surgical features and associated additional (or subsequent) predictions of 3D kinematic motion of a joint in accordance with one or more non-limiting implementations of the present technology.

FIG. 16 depicts a schematic diagram of a user interface for a selection of ranges of surgical features and associated boundaries of a refined prediction of 3D kinematic motion of a joint in accordance with one or more non-limiting implementations of the present technology.

### DETAILED DESCRIPTION OF THE INVENTION

The examples and conditional language recited herein are principally intended to aid the reader in understanding the principles of the present technology and not to limit its scope to such specifically recited examples and conditions. It will be appreciated that those skilled in the art may devise various arrangements which, although not explicitly described or shown herein, nonetheless embody the principles of the present technology and are included within its spirit and scope.

Furthermore, as an aid to understanding, the following description may describe relatively simplified implementations of the present technology. As persons skilled in the art would understand, various implementations of the present technology may be of a greater complexity.

In some cases, what are believed to be helpful examples of modifications to the present technology may also be set forth. This is done merely as an aid to understanding and, again, not to define the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Moreover, all statements herein reciting principles, aspects, and implementations of the present technology, as well as specific examples thereof, are intended to encompass both structural and functional equivalents thereof, whether they are currently known or developed in the future. Thus, for example, it will be appreciated by those skilled in the art that any block diagrams herein represent conceptual views of illustrative circuitry embodying the principles of the present technology. Similarly, it will be appreciated that any flowcharts, flow diagrams, state transition diagrams, pseudo-code, and the like represent various processes which may be substantially represented in computer-readable media and so executed by a computer or processor, whether or not such computer or processor is explicitly shown.

The functions of the various elements shown in the figures, including any functional block labeled as a “processor” or a “graphics processing unit”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. In one or more non-limiting implementations of the present technology, the processor may be a general purpose processor, such as a central processing unit (CPU) or a processor dedicated to a specific purpose, such as a graphics processing unit (GPU). Moreover, explicit use of the term “processor” or “controller” should not be construed to refer exclusively to hardware capable of executing software, and may implicitly include, without limitation, digital signal processor (DSP) hardware, network processor, application specific integrated circuit (ASIC), field programmable gate array (FPGA), read-only memory (ROM) for storing software, random access memory (RAM), and non-volatile storage. Other hardware, conventional and/or custom, may also be included.

Software modules, or simply modules which are implied to be software, may be represented herein as any combination of flowchart elements or other elements indicating performance of process steps and/or textual description. Such modules may be executed by hardware that is expressly or implicitly shown.

### 1. Overview of Example Technical Problem

Systems dealing with surgical training, surgery planning, and intra-operative (intra-op) visualization are mostly based on information obtained by reviewing medical imagery in static conditions and/or three-dimensional (3D) simulation (or estimation) based on static information. Such exams remain limited in terms of their capacity to evaluate various functional aspects of the different joints, and typically cannot be performed while the joint is moving (i.e., they are static in nature), whether actively or passively (e.g., active knee motion driven by a patient himself during walking, passive knee motion driven manually by a surgeon during a clinical exam).

Static systems are static by definition because they use planar scanning. If the joint moves outside of the plane, there is motion artefact. For example, a CT scan takes 15-60 seconds, so any movement during this time will superpose different images and add noise to the measure. Conventional systems are unable to successfully achieve accurate results while the joint is moving.

Active motion of a joint in weight-bearing (WB) conditions can also be collected using fluoroscopic data, but this approach is costly, invasive, and involves stronger radiations compared to X-rays. Newer fluoroscopic systems may improve these flaws and allow WB joint motion data collection (e.g., during sit up/down, step up/down, lunge, deep knee bend), but these are not feasible to assess walking, running, etc. In addition, generic models (e.g., anatomic) and biomechanical models (e.g., muscle activation, kinematics, loads, etc.) are also available but are not optimal as they are not patient-specific and may be time consuming.

Three-dimensional joint motion can also be estimated without motion data being collected. Visual estimations (e.g., conducted by a surgeon during a clinical exam, by a biomechanical specialist, etc.), or modelled estimations (e.g., motion patterns created by software models), can also be used to integrate a patient's joint mechanics in management care. While these estimations can be useful for global care and/or training purposes, their lack of accuracy and reliability constitute a limit to their use in surgical context.

Other existing methods quantify and display real-time passive behavior of a joint movement during surgery, while surgical staff move the joint in different directions. Although manual distractions are mostly done, tensioners may be used to apply consistent stress forces throughout passive movements of the joint. While it allows more standardization from one patient to another, the most appropriate amount of stress to apply for each patient is still unknown and, furthermore, it is not representative of what the joint can undergo in weight-bearing condition.

Computer-assisted testing such as digital tensioners may provide standardization. Such software indicates in real time and with precision to the user if they apply the right amount of stress. For example, in the CORI Digital Tensioner (Smith&Nephew), the tensioner applies a force between 70N and 80N if a green bar (showing the currently applied force) is within a white rectangle (showing the acceptable range of 70N to 80N). Other systems such as the Newton dynamic platform (Exactech) apply a quasi-constant distraction force of 90N per femorotibial compartment throughout a passive knee flexion/extension motion to assess knee balance under stress.

While it reduces the subjectivity of the staff, the correct amount of stress to be applied for each patient (and on each joint compartment, if applicable) to reproduce the stress occurring during active WB motion is still unknown because it is multifactorial as it depends on joint geometry, soft-tissue memory, contractures, laxity, muscle density, patient condition, and biomechanical profile. For example, two women of the same age/weight/arthritis-grading will not apply the same amount of stress on their knee, especially during WB activity.

Surgical planning, training, and intra-operative visualization should take into account the patient's active joint mechanics under dynamic and/or WB conditions. A technical problem that therefore exists is the absence of integration of the active dynamic joint alignment and kinematic motion (WB or not if applicable) within surgical planning, training and intra-operative interventions. Another technical problem that exists is that joint static measurements or passive motion, which are currently used in surgical interventions, poorly correlate with dynamic or active behavior. Furthermore, joint alignment typically changes throughout motion, making it more difficult to integrate in surgical procedures.

The weight-bearing case is considered differently because the non-weight-bearing function is already generally restored with standard surgery (i.e., tested through passive movement intraoperatively). For the use of ropes, elastics, tensor bands, etc., the same problem occurs: the amount of stress to exercise through elastics, etc. is unknown. Moreover, the direction of the force, or in this case the axis of the tensor bands, etc. is another parameter to consider.

Conventional systems have not resolved this technical problem. Physicians do not consider the 3D kinematic data (which may also be referred to as 3D active kinematic data) which makes it impossible to understand or visualize the impact of a surgical intervention on the kinematic motion (or active biomechanical behavior) of the joint, and the differences between the kinematic motion in dynamic conditions compared to static conditions or passive motion during surgery.

Surgeons can have access to tools measuring 3D kinematic data, but they do not have the software to process and present them in a way that is quickly understandable and actionable in their practice (e.g., in real time). Surgeons do not know how their intervention in the operating room will affect the joint behavior in weight-bearing conditions.

Surgeons mainly use bone morphology combined with lower limb imaging measures (e.g., alignment, joint line obliquity, etc.) to plan surgery and when technology is available, surgeons use passive kinematic information to adapt the surgical intervention. They do not take into consideration active dynamic motion nor the impact of WB conditions although these correspond to the state in which the joint will be more often exploited by the patient post-surgery. For example, in the operating room, surgeons mostly focus on balancing gaps (e.g., space between femoral and tibial implant) in total knee arthroplasty to prepare the knee so it can react in the best way possible to the loading forces in weight-bearing conditions (e.g., during walking). Therefore, there is a lack of functional active dynamic data to integrate data in WB conditions in care management to help surgeons/clinicians plan their intervention and make evidence-based decisions during surgery.

Another major deficiency is the fact that adjustments are often required intraoperatively. Physicians roughly address this issue by applying generic techniques which are not optimal for all patients.

In conventional approaches, physicians use techniques based on their beliefs and their “surgical philosophy” (e.g., mechanical alignment, kinematic alignment, restricted kinematic alignment, etc.). They can use different techniques to accommodate between different types of patients. These techniques aim at restoring knee function in the operating room, during passive movement of the knee in non-weight bearing conditions, or simply in full extension and at 90 degrees of flexion. It is not clear which technique would lead to the optimal outcome (i.e., the best knee function during active WB tasks) for each patient. As of 2024, 20% of patients were unsatisfied after knee replacement surgery and the American Academy of Orthopaedic Surgeons (AAOS) reported more than 700,000 knee replacement surgeries annually. Surgical interventions are not optimal for all patients.

### 2. Overview of Example Technical Solution

An example of a technical solution is an application to process 3D kinematic data, provide a simplified visualization to understand them, and illustrate for the surgeon the relationships between their intervention (e.g., a surgical intervention) and the joint dynamic behavior. This technical solution may be referred to as an application for generating a prediction of an impact of an intervention on 3D kinematic motion of a joint before or during an operation. This technical solution may also be referred to as a method for generating a prediction of an impact of an intervention on 3D kinematic motion of a joint before or during an operation. More generally, for example, when this technical solution involves more than one computing device, it may refer to as a system for generating a prediction of an impact of an intervention on 3D kinematic motion of a joint before or during an operation. For the purposes of simplicity, reference to a prediction of an impact of an intervention on 3D kinematic motion of a joint may be shortened to “a prediction of 3D kinematic motion”.

A method of implementing this technical solution comprises using 3D kinematic data from a functional evaluation (or kinematic/biomechanical profile/phenotype) of a joint during weight-bearing activity (or task) with a given system (e.g., a video camera, an accelerometer, an electromagnetic sensor, a gyroscope, an optical sensor, inertial sensor, etc.) preoperatively and merging these data with complementary surgical data related to the planned surgery (e.g., imaging, soft-tissue, clinical data, etc.). The merged data may be referred to as a merged data set.

Kinematic data may comprise, for example, angle, velocity, acceleration, displacement, amplitude, position, or any other metric which can be used to describe the motion of a body, segments(s), or system without consideration of the forces involved.

Although the embodiments of the invention described herein primarily discuss using 3D data, an implementation of this technical solution that uses 2D data is possible. This generalization includes, for example, one of more appropriate modifications. For example, where 3D data is referred to, it can be substituted by suitable 2D data (e.g., a plane orthogonal to the axis of rotation of the joint if it is a hinge joint). Also, for example, suitable modifications can be made to the embodiment to how the 2D data is used (e.g., using the planar data to predict the dynamic alignment of the joint).

From the merged data, patient-specific predictions of the 3D kinematic motion of the joint, described using functional parameters (e.g., dynamic alignment, biomechanical markers, functional concepts, functional scores, kinematic phenotype affiliation) are generated. Alternatively, or in addition, patient-specific simulations of the 3D kinematic motion of the joint are generated.

Initial predictions can be provided for kinematic phenotype affiliation, dynamic alignment behavior, biomechanical markers, and functional scores.

The visualization of the initial predictions can include text, graphics, charts, graphs, user interface elements (e.g., drop-down selections), etc. The visualization may include different sections, such as a global functional score, shortened list (e.g., three items) of what the surgeon should pay attention to, the WB task, biomechanical markers, indicators, and/or functional scores.

It is possible to visualize: (i) the initial prediction of the 3D kinematic motion of the human joint; and (ii) a refined prediction of the impact of a planned surgical intervention (e.g., implant type, size, resection measures, etc.) on the joint kinematic motion and functional parameters. For the purposes of simplicity, reference to a planned surgical intervention may be shortened to “an operation”.

The patient's 3D kinematic motion predictions (which may include the pattern difference between the dynamic and static joint alignment) in different steps of the surgical episode of care (e.g., pre-op planning, intra-op, post-op) are displayed.

The displays (or visualizations) of the predictions may provide the user(s) (e.g., surgeons, assistants, health care professionals), for example, a way to visualize the active dynamic behavior of the joint and the difference with the static joint alignment or manual testing results.

The user (e.g., clinician or surgeon) may prioritize the functional parameters that they want to be used for further refined predictions by setting lower and/or upper desirable boundaries on one or more functional parameters, eliminating parameters that they do not consider relevant for the outcome of the planned surgery, and/or use recommendations of the functional parameters to prioritize and their related boundaries to aim for optimal surgery based on the patient kinematic phenotype affiliation. The optimal surgery may be considered to be the one which maximizes the probability of achieving the best possible clinical outcomes post-op. Regarding the recommended functional parameters and their boundaries, they may correspond to patients belonging to the same kinematic phenotype which presented the best outcomes in studies that have been conducted (e.g., over a period of years).

### 3.0 Example Technical Solution for Knee Replacement Surgery

In the example of a knee replacement surgery, a surgeon and their team may meet in the weeks before the surgery and plan the surgery. Using different types of static imagery data (e.g., X-Ray, CT scans, etc.), they establish a pre-operative (pre-op) plan detailing what type of implant they will use (about a dozen types exist), which size (globally ranging from 1 to 10), and how they will position the implant components on the femur and on the tibia to achieve desired lower limb alignment (e.g., mechanical alignment, kinematic, functional, etc.). This preoperative plan is a road map which is destined to change during the next step in which the surgeon will have access to other types of information. Some robotic or digital surgical systems may allow intra-operative planning, in which case this step may be entirely done in the operating room (i.e., with no imagery done before).

During the intra-operative step, the surgeon is in the operating room. Once the knee is opened, the surgeon can use different techniques, such as cutting the bones according to the preoperative plan and then manually testing if the knee is balanced (i.e., testing if the knee is stable in full extension or at different flexion angles, or by assessing joint stability while flexing/extending the knee and applying lateral forces to mimic loading conditions) and adjusting bone cutting/resections and soft-tissue releasing or adjusting implant or bearing size during a trial-and-error process until they are satisfied.

A more advanced technique involves using computer-assisted technologies (e.g., navigation system or surgical robot) which accurately registers the localization of the bones in 3D and provides additional information to the surgeon on a monitor. One example is the balance gap, which is the distance between the femur and the tibia on the internal or external side of the knee. With this kind of technology, the surgeon can select a virtual implant (type, size) and position it on the bone model on the monitor. The surgeon will see the balance gaps and the bone cuts evolve while moving the implant in different positions. This is the intra-operative planning where the surgeon starts from the preoperative plan and refines it virtually until they are satisfied regarding the surgery planned outcomes (e.g., planned balance, planned lower-limb alignment, etc.). Then they proceed to the actual bone cutting and implant insertion.

The last step is post-operative (post-op), for example, after the surgery. Even if the surgery was a “technical success” (i.e., the damaged bone structures have been removed, the knee and lower limb have been realigned, the knee moved smoothly and/or was stable while the surgeon tested it intraoperatively during a passive movement, no implant loosening or migration, no infection or allergic reaction, etc.), patients may report functional limitations, specifically during weight-bearing activities. It can partially be explained by the fact that the complete surgical process has been done without using any information on how the knee actively behaved or would behave under loading, during a weight-bearing activity. In this post-operative step, patients can follow conservative care programs (e.g., exercises, bracing, etc.) to address these residual functional limitations, or in worst case scenarios they can have revision surgery.

### 3.1 Pre-Op Visualizations for Knee Replacement Surgery

The pre-op visualizations may include different sections, such as a global functional score, shortened list (e.g., three items) of which functional parameters the surgeon should pay attention to, the WB task, biomechanical markers, indicators, functional concepts, and/or other functional scores.

The global functional score may be for a particular WB task such as gait. The score may be a numerical score, such as score out of 100 (e.g., 54 out of 100).

The shortened list of which functional parameters the surgeon should pay attention to may include one or more biomechanical markers of interest and an associated quantity/measurement, the affiliated kinematic phenotype for the patient, an evaluation of predisposing risk factors of pain after total knee arthroplasty (TKA), a classification type related to a functional concept, etc. For example, one of the biomechanical markers may be varus thrust at 3.6 degrees. Another biomechanical marker may be dynamic flexion contracture at 18 degrees. The kinematic phenotype may be a label such as ‘1’ (or ‘A’). The predisposing risk factor of pain after TKA may be labelled as one of low, moderate, or high (either discretely or on a continuum). The classification type related to a functional concept may be, for example, medial pivot. Another classification type related to a functional concept may be, for example, a varus-to-neutral type (or labelled as ‘type 12’) of dynamic coronal plane alignment of the knee from static alignment (or “dynamic CPAK”).

The WB task may be walking, sitting down, standing up, going up and down stairs or a stepper unit, squatting, lunging, leg pressing, hopping, jumping, or running. Alternatively, the WB task may be shown in categories, such as gait.

The biomechanical markers may include functional hip-knee-ankle angle (fHKA) (e.g., 7.1 degrees), varus heel strike angle (e.g., 9.2 degrees), varus minimum angle (e.g., 3.7, degrees), mean varus-valgus angle (e.g., 2.7 degrees), varus thrust angle (e.g., 3.6 degrees), expected varus, and kinematic phenotype (e.g., ‘1’).

The indicators may include frontal plane (by phenotype), frontal plane dynamic alignment, etc. For example, for the frontal plane indicator, there may be an associated graph of frontal plane behavior during gait for phenotype 1, showing values between 0 degrees and 15 degrees (e.g., mostly between 5 degrees and 10 degrees). Also for example, for the dynamic alignment indicator, there may be an associated (one-dimensional) graph of frontal plane dynamic alignment, showing values between- 5 degrees and 15 degrees. The indicators may also include functional parameters during NWB tasks (e.g., active NWB flexion, passive NWB flexion, etc.).

The functional concepts may include pivot pattern during stance (e.g., ‘lateral pivot’), multi-pivot pattern during gait (e.g., ‘yes’), type of dynamic varus-valgus alignment of the knee (DVAK) from static alignment (e.g., ‘type 23’). These concepts may be associated with illustrations (e.g., using representations of a neutrally aligned knee in static and of a valgus aligned knee in dynamic to illustrate a DVAK type 23, presenting the anterior/posterior displacement of the medial and/or lateral femoral condyle or epicondyle to illustrate a pivot pattern).

The functional scores may include joint functional scores, such as loading (e.g., 55%), stiff knee (e.g., 75%), gait speed (e.g., 100%), frontal plane behavior (e.g., 56%), medial compartment stress (e.g., 25%), and lateral compartment stress (e.g., 78%). These scores may be displayed graphically (e.g., using a radial graph).

The functional evaluation can include using kinematic data of a human joint (e.g., knee, hip) during different types of WB tasks (i.e., walking, sitting down, standing up, stepping up or down stairs, squatting, lunging, leg pressing, hopping, jumping, running, etc.) in close or open kinematic chain.

### 3.2 Intra-Op Visualizations for Knee Replacement Surgery

The intra-op visualizations may include different sections, such as a front view of the top part of the knee (e.g., femur), a front view of the bottom part of the knee (e.g., tibia), a top view of the top part of the knee (e.g., femur), a top view of the bottom part of the knee (e.g., tibia), a femoral implant component size, a tibial implant component size, and a bearing insert (or bearing polyethylene) thickness and design, as well as selections for functional parameters (e.g., flexion loading angle, varus thrust angle, pivot pattern during stance, etc.), and a kinematic phenotype affiliation, which may show related information and/or graphs when selected.

The front view of the top part of the knee may indicate its alignment (e.g., component valgus), show an angle (e.g., 0 degrees), and show femoral resection measures on each compartment (e.g., 7 mm medial and 6 mm lateral). The front view of the bottom part of the knee may indicate its alignment (e.g., component valgus), show an angle (e.g., 0 degrees), and show tibial resection measures on each compartment (e.g., 12 mm medial and 8.5 mm lateral). The top view of the top part of the knee may show femoral posterior resection measures on each compartment (e.g., 9.5 mm medial and 12 mm lateral). The top view of the bottom part of the knee may show just the localization of the tibial implant.

The femoral implant component size may show a standardized size (e.g., size 5). The tibial implant component size may show a standardized size (e.g., size 4). The polyethylene thickness may show an actual thickness (e.g., 9 mm).

When flexion loading angle is selected, a flexion loading angle may be shown. For example, the angle may be 11 degrees, and a graph may be generated that shows where the value of this angle lies within different ranges, such as healthy or unhealthy (e.g., inferior to 5 degrees corresponding to unhealthy flexion loading angle values, superior to 15 degrees corresponding to healthy values). An additional graph may be generated to show more details (e.g., the global flexion angle graph throughout the WB task).

When kinematic phenotype is selected, the affiliated phenotype may be described (e.g., mean values of functional parameters for knees affiliated with this phenotype). Functional parameters may be shown along with specific angles, such as flexum heel strike (e.g., 12 degrees) and varus alignment maximum (e.g., 9.6 degrees). A graph may be generated that shows frontal plane behavior during the WB task for the phenotype (e.g., phenotype 1).

When varus thrust angle is selected, the varus thrust angle may be shown. For example, the angle may be 2.55 degrees, and a graph may be generated that shows where the angle lies within different ranges, such as good, bad, worst (e.g., 0 to 1.5 degrees is good, 1.5 to 2.5 degrees is bad, and 2.5 to 3.5 degrees is worst). An additional graph may be generated to show more details.

When pivot pattern during stance is selected, the pivot pattern may be shown. For example, the pattern may be ‘medial pivot’, and an illustration may be generated that shows where the center of rotation lies on a diagram of a tibial plateau within different zones, such as medial, lateral, extra-articular (e.g., medial pivot if the center of rotation is located between the left edge of the tibial plateau and its center, extra-articular if the center of rotation is located outside of the edges of the tibial plateau). An additional graph may be generated to show more details (such as the anterior/posterior displacement of the medial and/or lateral femoral condyle or epicondyle).

When type of DVAK from static alignment is selected, the DVAK type may be shown. For example, the type may be ‘type 23’, and an illustration may be generated that shows a representation of a neutrally aligned knee corresponding to the static condition and a representation of a valgus aligned knee corresponding to the dynamic condition. An additional graph may be generated to show more details (such as the categories of the DVAK classification with their numerical grading with or without the varus-valgus angle in static condition and/or the maximum/minimum varus-valgus angle reached in dynamic condition).

### 3.3 Planned Surgical Interventions for Knee Replacement Surgery

This technical solution may be used pre-operatively for surgery planning and surgery training, or during surgery for the user to visualize the prediction of the impact of a planned surgical intervention.

There may be a large number (e.g., over 1000) possible surgical interventions for a particular joint. This is because a “planned surgical intervention” is the sum of all surgical features set at specific values. So two surgical interventions would be different if all their surgical features were the same except one by the smallest margin possible.

For example, these three planned surgical interventions for a posterior-stabilized (PS) implant are all different:
(1) PS implant, femoral implant component size 3, tibial implant component size 5, femoral resections 6.5-7.5 mm, tibial resections 5.0-5.5 mm;
(2) PS implant, femoral implant component size 3, tibial implant component size 5, femoral resections 7.0-7.5 mm, tibial resections 5.0-5.5 mm;
(3) PS implant, femoral implant component size 3, tibial implant component size 4, femoral resections 7.0-7.5 mm, tibial resections 5.0-5.5 mm.

In these three planned surgical interventions, the second is different from the first because of the different femoral resections, and the third is different from the first two because of the different tibial implant component size.

The prediction of the impact of a planned surgical intervention can be visualized to assist the user. For example, there may be two screens (or two windows, or two views) from which one can switch back and forth. The first screen (or window or view) may have details of the planned surgical intervention and a summary of the corresponding predicted functional parameters. The second screen (or window or view) may show information for the user to visualize the prediction in detail. It should be appreciated that any reference to a screen in the embodiments described herein may be replaced with a window (e.g., shown on a screen) or a view (e.g., a visual interface element on any display device, screen, or otherwise).

The first screen may include information on the type and size of the implant components, surgical features relative to the femur, surgical features relative to the tibia, soft-tissue release information, and a summary of the prediction of the impact of the selected intervention on functional parameters.

With regards to the implant, the type may be PS, and the size may be for the femoral component ‘3’ and for the tibial component ‘5’.

The surgical features for the femur may include post-op alignment (e.g., 3 degrees varus), medial resection (e.g., 6.5 mm), lateral resection (e.g., 7.5 mm), medial posterior resection (e.g., 4.5 mm), and lateral posterior resection (e.g., 4.0 mm).

The surgical features for the tibia may include post-op alignment (e.g., 1 degree valgus), medial resection (e.g., 5.5 mm), lateral resection (e.g., 5.5 mm), and slope (e.g., 3 degrees).

The soft-tissue release information may include a specific ligament (e.g., medial collateral ligament—MCL) and the type of release (e.g., posterior capsule).

The summary of the prediction of the impact of the selected intervention on functional parameters may include predicted values with or without ranges for functional alignment (e.g., 3 degrees+/−1 degree), dynamic varus maximum (e.g., 4 degrees+/−1 degree), dynamic varus minimum (e.g., 1 degree+/−1 degree), varus thrust (e.g., 3.6 degrees), flexion contracture (e.g., 18 degrees), functional score (e.g., 54), kinematic phenotype (e.g., 1), static flexum (e.g., 12 degrees), flexum at heel contact (e.g., 18 degrees), flexum during stance (e.g., 19 degrees), total range of motion (e.g., 40.5 degrees), flexion loading (e.g., 11 degrees).

The second screen may include different sections, such as a global functional score, shortened list (e.g., three items) of what the surgeon should pay attention to, the WB task, biomechanical markers, indicators, functional concepts, and/or functional scores.

The global functional score may be for a particular functional task such as global knee functional score. The score may be a numerical score, such as score out of 100 (e.g., 54 out of 100).

The shortened list of what the user should pay attention to may include one or more biomechanical markers of interest and an associated quantity/measurement, the affiliated kinematic phenotype for the patient, an evaluation of predisposing risk factors of pain after total knee arthroplasty (TKA), a classification type related to a functional concept, etc. For example, one of the biomechanical markers may be varus thrust at 3.6 degrees. Another biomechanical marker may be dynamic flexion contracture at 18 degrees. The kinematic phenotype may be a label such as ‘1’ (or ‘A’). The predisposing risk factor of pain after TKA may be labelled as one of low, moderate, or high (either discretely or on a continuum). The classification type related to a functional concept may be, for example, medial pivot. Another classification type related to a functional concept may be, for example, a varus-to-neutral type (or labelled as ‘type 12’) of dynamic coronal plane alignment of the knee from static alignment (or “dynamic CPAK”).

The WB task may be walking, sitting down, standing up, going up and down stairs or a stepper unit, squatting, lunging, leg pressing, hopping, jumping, or running. Alternatively, the WB task may be shown in categories, such as gait.

The biomechanical parameters may include static flexum (e.g., 12 degrees), flexum at heel contact (e.g., 18 degrees), flexum during stance (e.g., 19 degrees), total range of motion (e.g., 40.5 degrees), flexion loading (e.g., 11 degrees), and kinematic phenotype (e.g., ‘1’).

The indicators may include sagittal plane behavior, frontal plane behavior, joint behavior during NWB tasks, etc. For example, for frontal plane behavior, there may be an associated graph of sagittal plane behavior, showing flexion values between 0 and 70 degrees (e.g., mostly between 20 and 60 degrees).

The functional concepts may include pivot pattern during stance (e.g., ‘lateral pivot’), multi-pivot pattern during gait (e.g., ‘yes’), type of dynamic varus-valgus alignment of the knee (DVAK) from static alignment (e.g., ‘type 23’). These concepts may be associated with illustrations (e.g., using representations of a neutrally aligned knee in static and of a valgus aligned knee in dynamic to illustrate a DVAK type 23).

The functional scores may include gait functional scores, such as loading (e.g., 55%), stiff knee (e.g., 75%), gait speed (e.g., 100%), sagittal plane behavior (e.g., 56%), medial compartment stress (e.g., 25%), and lateral compartment stress (e.g., 78%). These scores may be displayed graphically (e.g., using a radial graph).

### 3.4 Real-Time Feedback for Knee Replacement Surgery

This technical solution may be useful in different scenarios pre-operatively for surgery planning and surgery training, or during surgery to get real-time feedback to select the optimal surgery to restore joint function for each patient.

During surgery, it may be helpful for the surgeon to visualize the impact of modifying a surgical feature (e.g., cut 7.0 mm on the tibial lateral side instead of 6.5 mm) on a surgery outcome (e.g., the planned balance gap) in a short period of time (e.g., under seconds). It allows the surgeon to test a multitude of feature combinations until they reach the one they are satisfied with, without spending extra minutes (or even hours) in the operating room. This technical solution may provide guidance in a short timeframe (e.g., in real time). For example, if one surgical feature is modified, the surgeon will see the refined predictions (e.g., the predicted 3D weight-bearing behavior of the knee) within a few seconds. This may be the time needed to run the algorithm with a new input (e.g., select a 7.0 mm cut and display the refined prediction).

A server, for example, may execute the required programs (or “algorithms”) on real-time data and make updates as the surgery progresses. Optionally, at any time, the surgeon may intervene and change selected features, functional parameters priorities, and/or functional parameters boundaries, as well as add data collected during the surgery (which can then be used as inputs).

### 3.5 Possible Additional Features

The technical solution may include visualizing (e.g., graphical representations, augmented reality, etc.) of predictions on different types of displays and/or assisted surgery equipment (e.g., surgical planning software or systems, headsets, glasses, visual assistive aids, robotic systems, navigation system, holographic technology, etc.). These may be used in combination or in absence of medical imagery data (e.g., CT scans, MRI, X-ray, etc.).

The visualizations of the predictions may be the same in the absence or presence of medical imagery data and may be the same regardless of the equipment used because they are in the form of a graphical guide plus a narrative on a standard bone model. In the case where 3D medical imagery data are available (e.g., CT scan, EOS imaging, ultrasound, etc.), these may be displayed in replacement of the standard bone model.

The technical solution may include the ability to input complementary surgical data collected during surgery, for example, manually by a surgeon/assistant or collected with computer-assisted technologies (a surgical robot, navigation system, etc.) to adjust kinematic motion predictions in real time (or in a short period of time such as under 2 seconds). This intra-op data may include, for example, joint (frontal plane) alignment data during passive motion, soft-tissue behavior data, balance gap during passive movement, degrees of flexion contracture, joint frontal alignment at discrete degrees of flexion, anatomical information (e.g., posterior slope, joint line, native deformity, axes like transepicondylar axis relative position, posterior condylar angle, transepicondylar axis relative position vs. distal bone, flexion/extension axis), etc.

The user (e.g., surgeon, assistant) may make changes during the surgery, without having to stop anything because it is integrated in the process just like the use of computer-assisted technologies (e.g., navigation system or surgical robots)—the user may take some time to identify bone landmarks for the computer-assisted technologies to properly function, which may be just part of the surgery. The surgeon may use a microphone as input, for example, to allow the surgeon to continue using their hands during the surgery while interacting with the application (or algorithms).

The technical solution may include post-op analysis and/or recommendations, for example, immediately after a surgical intervention or even months later. The post-op analysis and/or recommendations may explore what was predicted for a specific case and/or re-simulate post-op recommendations.

### Computing Device

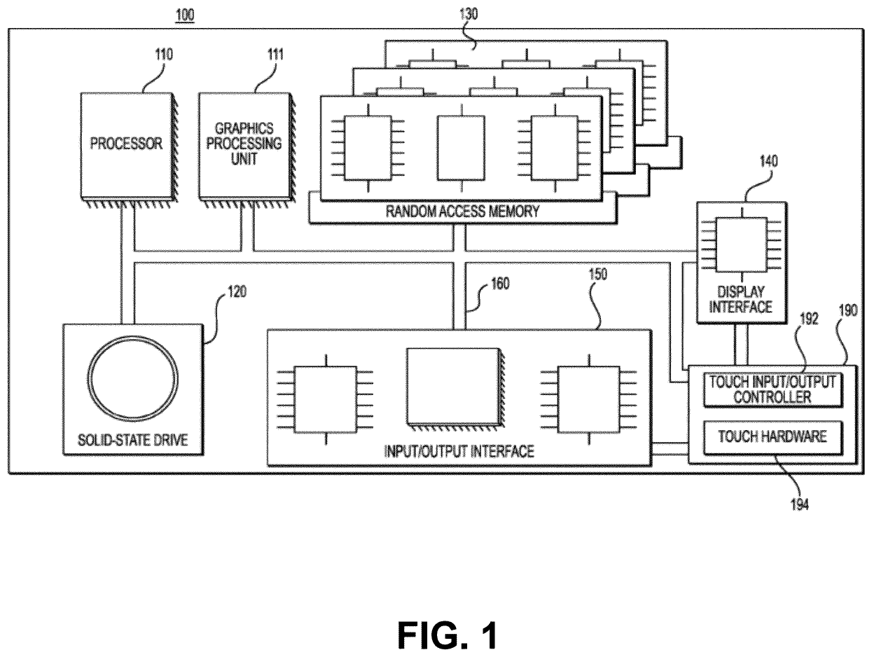

Referring to FIG. 1, there is shown a computing device 100 suitable for use with some implementations of the present technology, the computing device 100 comprising various hardware components including one or more single or multi-core processors collectively represented by processor 110, a graphics processing unit (GPU) 111, a solid-state drive 120, random-access memory 130, a display interface 140, and an input/output interface 150.

Communication between the various components of the computing device 100 may be enabled by one or more internal and/or external buses 160 (e.g., a PCI bus, universal serial bus, IEEE 1394 “Firewire” bus, SCSI bus, Serial-ATA bus, etc.), to which the various hardware components are electronically coupled.

The input/output interface 150 may be coupled to a touchscreen 190 and/or to the one or more internal and/or external buses 160. The touchscreen 190 may be part of the display. In one or more implementations, the touchscreen 190 is the display. The touchscreen 190 may equally be referred to as a screen 190. In the implementations illustrated in FIG. 1, the touchscreen 190 comprises touch hardware 194 (e.g., pressure-sensitive cells embedded in a layer of a display allowing detection of a physical interaction between a user and the display) and a touch input/output controller 192 allowing communication with the display interface 140 and/or the one or more internal and/or external buses 160. In one or more implementations, the input/output interface 150 may be connected to a keyboard (not shown), a mouse (not shown), or a trackpad (not shown) allowing the user to interact with the computing device 100 in addition or in replacement of the touchscreen 190. In one or more implementations, the input/output interface 150 is part of a robotic system where there is a screen 190 for one user (e.g., a surgeon) and a second screen (not shown) for a robot product specialist to enter data on.

According to implementations of the present technology, the solid-state drive 120 stores program instructions suitable for being loaded into the random-access memory 130 and executed by the processor 110 and/or the GPU 111 for an application for generating a prediction of the impact of an intervention on 3D kinematic motion of a joint before or during an operation. For example, the program instructions may be part of a library or an application.

The computing device 100 may be implemented as a server, a desktop computer, a laptop computer, a tablet, a smartphone, a personal digital assistant, or any device that may be configured to implement the present technology, as it may be understood by a person skilled in the art.

### System

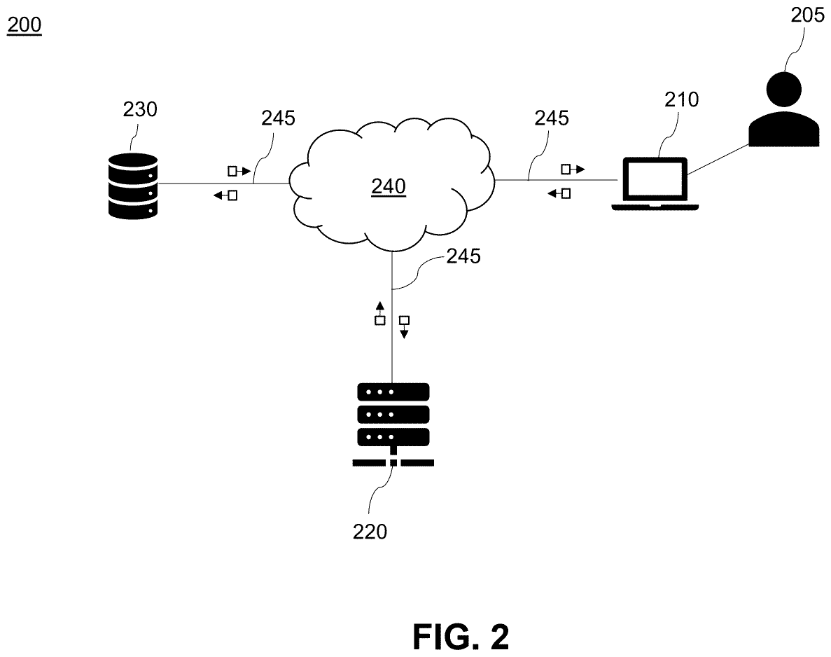

Referring to FIG. 2, there is shown a schematic diagram of a system 200, the system 200 being suitable for implementing one or more non-limiting implementations of the present technology. It is to be expressly understood that the system 200 as shown is merely an illustrative implementation of the present technology. Thus, the description thereof that follows is intended to be only a description of illustrative examples of the present technology. This description is not intended to define the scope or set forth the bounds of the present technology. In some cases, what are believed to be helpful examples of modifications to the system 200 may also be set forth below. This is done merely as an aid to understanding, and, again, not to define the scope or set forth the bounds of the present technology. These modifications are not an exhaustive list, and, as a person skilled in the art would understand, other modifications are likely possible. Further, where this has not been done (i.e., where no examples of modifications have been set forth), it should not be interpreted that no modifications are possible and/or that what is described is the sole manner of implementing that element of the present technology. As a person skilled in the art would understand, this is likely not the case. In addition, it is to be understood that the system 200 may provide in certain instances simple implementations of the present technology, and that where such is the case they have been presented in this manner as an aid to understanding. As persons skilled in the art would understand, various implementations of the present technology may be of a greater complexity.

In one or more implementations of the present technology, the system 200 is the system for generating a prediction of 3D kinematic motion of a joint before or during an operation. In one or more implementations of the present technology, the system 200 is part of the system for generating a prediction of 3D kinematic motion of a joint before or during an operation. In one or more implementations of the present technology, the system for generating a prediction of 3D kinematic motion of a joint before or during an operation is part of the system 200.

The system 200 comprises inter alia a client device 210 associated with a user 205, a server 220, and a database 230 communicatively coupled over a communications network 240. Alternatively, the system 200 may comprise some, but not all, of these components. For example, the system 200 may comprise a client device 210 associated with a user 205, and a database 230 communicatively coupled with the client device 210.

### Client Device

The system 200 comprises a client device 210. The client device 210 is associated with the user 205. As such, the client device 210 can sometimes be referred to as a “computing device”, “end user device”, or “client computing device”. It should be noted that the fact that the client device 210 is associated with the user 205 does not need to suggest or imply any mode of operation—such as a need to log in, a need to be registered, or the like. The client device 210 may be one particular device from amongst a plurality of client devices within the system 200.

The client device 210 comprises one or more components of the computing device 100 such as one or more single or multi-core processors collectively represented by the processor 110, the graphics processing unit (GPU) 111, the solid-state drive 120, the random-access memory 130, the display interface 140, and the input/output interface 150.

It will be appreciated that the client device 210 may be implemented as a server, a desktop computer, a laptop, a smartphone, and the like, or within a digital surgical system. The client device 210 may be implemented as a virtual machine, for example, when the system 200 runs in a cloud infrastructure, or when it is embedded in a larger system (e.g., digital surgical system). Non-limiting examples of the given digital surgical system within which the client device 210 may be implemented include MAKO (Stryker), CORI (Smith&Nephew), VELYS (Johnson&Jonhson-DePuy-Synthes), NextAR (MedActa), ROSA (Zimmer-Biomet), Orthopilot (Aesculap), GPS (Exactech), TMINI (Think-Surgical), Knee+AR (Pixee-Medical), and Arvis (Enovis).

In one or more implementations, the client device 210 is configured to execute an application (e.g., a desktop application, a browser application, a smartphone app, etc.) for generating a prediction of 3D kinematic motion of a joint before or during an operation. The purpose of the given browser application is to enable the user of the prediction generator to access one or more web resources. How the given browser application is implemented is not particularly limited. Non-limiting examples of the given browser application that is executable by the client device 210 include Google™ Chrome™, Mozilla™ Firefox™, Microsoft™ Edge™, and Apple™ Safari™.

In one or more implementations, the user 205 may be an operator of the system for generating a prediction of 3D kinematic motion of a joint before or during an operation. The user 205 may be an agent (e.g., an intelligent agent) or a bot carrying out the role of the operator. The user 205 may be a person using a user device connected to the system for generating a prediction of 3D kinematic motion of a joint before or during an operation. The user 205 may be an external user device, such as one that is external to the system for generating a prediction of 3D kinematic motion of a joint before or during an operation.

### Server

The server 220 is configured to inter alia: (a) communicate with the client device 210 and/or other hardware or software described herein to implement an application for generating a prediction of 3D kinematic motion of a joint before or during an operation; (b) to control some or all of the hardware, devices, and/or circuitry to implement the application; and/or (c) execute some or all of the programs or algorithms to implement the application. The server 220 may be connected to the Internet. Alternatively, the server 220 may be a standalone computer, for example, for use with (or as part of) a digital surgical system that is not connected to the Internet.

How the server 220 is configured to do so will be explained in more detail herein below.

It will be appreciated that the server 220 can be implemented as a conventional computer server and may comprise at least some of the features of the computing device 100 shown in FIG. 1. In a non-limiting example of one or more implementations of the present technology, the server 220 is implemented as a server running an operating system (OS). Needless to say that the server 220 may be implemented in any suitable hardware and/or software and/or firmware or a combination thereof. In the disclosed non-limiting implementation of the present technology, the server 220 is a single server. In one or more alternative non-limiting implementations of the present technology, the functionality of the server 220 may be distributed and may be implemented via multiple servers (not shown).

The implementation of the server 220 is well known to the person skilled in the art. However, the server 220 comprises a communication interface (not shown) configured to communicate with various entities (such as the database 230, for example, and other devices potentially coupled to the communication network 240) via the communication network 240. The server 220 further comprises at least one computer processor (e.g., the processing device of the computing device 100) operationally connected with the communication interface and structured and configured to execute various processes to be described herein.

### Database

A database 230 is communicatively coupled to the server 220 and the client device 210 via the communications network 240 but, in one or more alternative implementations, the database 230 may be directly coupled to the server 220 without departing from the teachings of the present technology. Although the database 230 is illustrated schematically herein as a single entity, it will be appreciated that the database 230 may be configured in a distributed manner; for example, the database 230 may have different components, each component being configured for a particular kind of retrieval therefrom or storage therein. Alternatively, or in addition, the database 230 may represent two or more databases; for example, the database 230 may represent a first database (e.g., for storing one category of data) and a second database (e.g., for storing another category of data).

The database 230 may be a structured collection of data, irrespective of its particular structure or the computer hardware on which data is stored, implemented or otherwise rendered available for use. The database 230 may reside on the same hardware as a process that stores or makes use of the information stored in the database 230, or it may reside on separate hardware, such as on the server 220. The database 230 may receive data from the server 220 for storage thereof and may provide stored data to the server 220 for use thereof.

In one or more implementations of the present technology, the database 230 is configured to store inter alia: (i) data produced or received by the hardware, software, or circuitry that implement the application (or system) for generating a prediction of 3D kinematic motion of a joint before or during an operation; and/or (ii) the programs and/or algorithms that implement the application (or system).

### Communication Network

In one or more implementations of the present technology, the communications network 240 is the Internet. In one or more alternative non-limiting implementations, the communication network 240 may be implemented as any suitable local area network (LAN), wide area network (WAN), a private communication network, or the like. It will be appreciated that implementations for the communication network 240 are for illustration purposes only. How a communication link 245 (not separately numbered) between the client device 210, the server 220, the database 230, and/or another computing device (not shown) and the communications network 240 is implemented will depend inter alia on how each computing device is implemented.

The communication network 240 may be used in order to transmit data packets amongst the different parts of the system 200. The communication network 240 may be used to transmit requests between the hardware, software, or devices used to implement the system for generating a prediction of 3D kinematic motion of a joint before or during an operation.

### Method for Generating a Prediction of 3D Kinematic Motion of a Joint Before or During an Operation

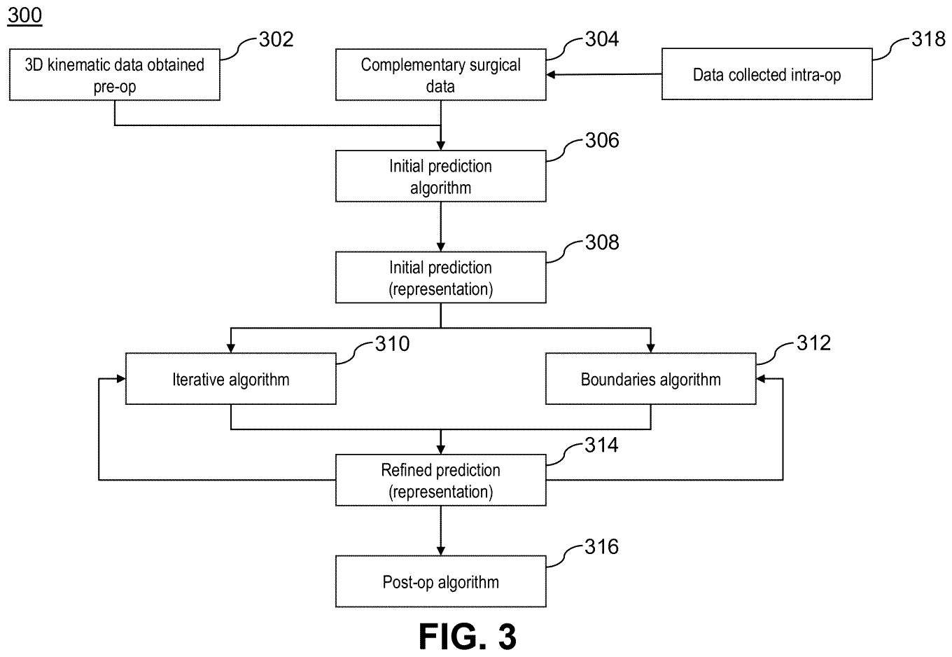

FIG. 3 depicts a flowchart of a method 300 for generating a prediction of 3D kinematic motion of a joint before or during an operation in accordance with one or more non-limiting implementations of the present technology. The method 300 may be used for generating and displaying 3D kinematic motion of a joint during weight-bearing (WB) activity (before and/or during surgery) as well as the predicted impact of a surgical intervention on this kinematic motion.

In one or more implementations, the server 220 comprises a processing device such as the processor 110 and/or the GPU 111 operatively connected to a non-transitory computer readable storage medium such as the solid-state drive 120 and/or the random-access memory 130 storing computer-readable instructions. The processing device, upon executing the computer-readable instructions, is configured to or operable to execute the method 300.

The method 300 begins at one or more of processing steps 302, 304, or 318. For example, the method 300 may begin with processing step 302 then continue with another processing step. Also, for example, the method 300 may begin with processing step 302 then continue with processing step 304, before proceeding to another processing step. Also, for example, the method 300 may begin with processing steps 302 and 304 being executed in combination, independently, or in parallel. Also, for example, the method 300 may begin with processing step 318 before proceeding with another processing step, such as processing step 302.

According to processing step 302, the processing device receives 3D kinematic data obtained pre-op (i.e., before an operation). The 3D kinematic data may relate to the 3D kinematic motion during a weight-bearing action or during the weight-bearing action in combination with motion during a non-weight-bearing action. The 3D kinematic data may be received, for example, from a “capturing system”. The capturing system may be any system which can capture 3D kinematic data, such as a video camera, stereo camera, Light Detection and Ranging (LiDAR) sensor, gyroscopic sensor, an inertial measurement unit, an accelerometer, a markerless motion capture system, an electromagnetic sensor, a gyroscope, an optical sensor, a radiographic system, a fluoroscopic imaging system, etc. or any motion sensor used to obtain 3D kinematic data of one or more joints. Alternatively, or in addition, the processing device may receive a biomechanical profile, such as a kinematic phenotype affiliation, which can be identified using other (potentially less accurate) 3D kinematic assessment devices. In such a case, the average 3D kinematic data related to this phenotype may be used as input. Alternatively, or in addition, the data includes kinematic data from a joint during different functional weight-bearing tasks such as walking, squatting, sitting, climbing up stairs, etc. The data may also include kinematic data from a joint during different functional non-weight-bearing tasks such as seated leg extensions, hamstring curl, passive or active range-of-motion exercises, etc.

In at least one implementation, the 3D kinematic data obtained pre-op in processing step 302 may be 3D motion estimations (e.g., visual estimations, modelled estimations, etc.) instead of (or in addition to) 3D kinematic data collected from a capturing system.

According to processing step 304, the processing device receives complementary surgical data. The complementary surgical data may relate to the joint and/or the operation. The complementary surgical data may be received from a capturing system or from user input related to the planned surgery. The complementary surgical data may be obtained, for example, from medical records or examinations (physical/clinical/socio-demographic data) or from assessments tools (e.g., 2D or 3D imaging data like X-Ray or CT scan, navigation system, surgical robot, etc.). The complementary surgical data may include one or more of: 2D imaging data, 3D imaging data, data from image-based and/or imageless surgical robotic-assisted systems or other computer-aided surgical systems, such as, but not limited to, surgical features, bony morphology, static joint alignment data, dynamic joint alignment data during passive movement, joint kinematic data during passive movement, soft-tissue behavior data (e.g., laxity gaps), physical data, clinical data, or socio-demographic data. The complementary surgical data may include data collected intra-op (i.e., during the operation, intraoperative parameters), which may come from processing step 318.

If the complementary surgical data is obtained from medical records or examinations, they may be input manually by the user and the format may be imposed (e.g., hip-knee-ankle angle on standing X-Ray: XXX.XX°; Sex: Male or Female, etc.).

If the complementary surgical data is obtained from assessments tools, they can also be input manually. This may be the case if the surgeon uses a computer-assisted surgical system (e.g., surgical robot) and can see the value of a measurement on a monitor and inputs it manually in the system (e.g., knee alignment deformity at 30 degrees of passive flexion with varus stress: XX.XX°).

If an assessment tool such as a computer-assisted surgical system is used, then the complementary surgical data may be processed within so it can follow the same imposed format. For example, if a surgeon uses a surgical robot: while the surgeon moves the knee through a passive movement, the robot collects balance gap data and processes it so it can be input in the system as “gap at 0 degrees is 2.5 mm, gap at 10 degrees is 2 mm, gap at 20 degrees is 1.5 mm, etc.”.

If the complementary surgical data is obtained intra-op (e.g., as described in processing step 318), this may be used to produce a new initial prediction in processing step 306.

In at least one implementation, the processing device, after receiving the complementary surgical data, merges the 3D kinematic motion data and the complementary surgical data, thereby creating a merged data set.

In at least one implementation, the processing device derives, from the merged data set, one or more functional parameters (e.g., biomechanical markers, functional concepts, kinematic phenotype affiliation, etc.) characterizing the 3D kinematic motion.

According to processing step 306, the processing device runs an initial prediction algorithm using the 3D kinematic data and the complementary surgical data. In at least one implementation, the processing device may run the initial prediction algorithm using the 3D kinematic data without complementary surgical data. The initial prediction algorithm generates an initial prediction. The initial prediction may be an initial prediction of the 3D kinematic motion before or during the operation based on one or more functional parameters. The processing device may, for example, send data through a server where an algorithm merges the data, then calculates and executes (i) the initial prediction of the 3D kinematic motion (e.g., functional parameters such as biomechanical markers, functional concepts, kinematic phenotype affiliation, etc.) based on the merged data and (ii) the refined predictions of the impact of the planned surgical intervention based on selected surgical features (e.g., implant type, size, soft-tissue balancing, slope, other intraoperative parameters, etc.) on the 3D joint kinematic motion/functional parameters. The merged data may be referred to as a merged data set. The processing device may require a minimum amount of data for specific categories, for example, a minimum of two surgical features (e.g., implant type, implant positioning).

If the complementary surgical data is obtained intra-op (e.g., as described in processing step 318), the initial prediction algorithm may be run with a new set of complementary data including intra-op data as inputs so that a new initial prediction is calculated.

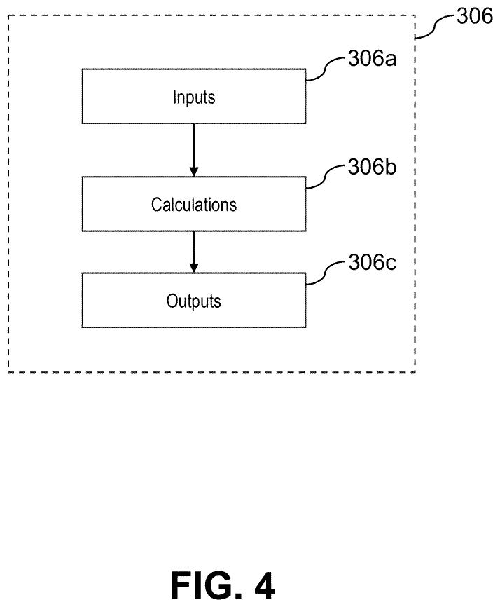

In at least one implementation, the processing step 306 generates predictions using the initial prediction algorithm shown in FIG. 4, using processing steps 306a, 306b, and 306c.

According to processing step 306a, the initial prediction algorithm takes as input 3D kinematic data and the complementary surgical data. The complementary surgical data may optionally include data collected intra-op (e.g., as described in processing step 318) if available. In at least one implementation, the initial prediction algorithm takes as input 3D kinematic data without complementary surgical data.

According to processing step 306b, the initial prediction algorithm performs calculations using the input from processing step 306a. These calculations may include one or more of: (1) processing all data inputs; (2) extracting functional parameters from 3D kinematic weight-bearing data; (3) calculating functional concepts and/or functional scores (ruled-based); (4) identifying kinematic phenotype affiliation; and (5) rules-based estimation of joint soft-tissue behavior and ligamentous laxities.

According to processing step 306c, the initial prediction algorithm generates a display of the initial prediction based on the calculations performed in processing step 306b. This display may include one or more of: (1) all calculated parameters; and (2) graphics including pattern difference between static and dynamic joint alignment, pattern difference between parameters during weight-bearing vs. non-weight bearing activities, or pattern difference between parameters during passive vs. active movement (weight-bearing and non-weight bearing) of the joint. In at least one implementation, the processing device generates a graphical output (e.g., in real time) before or during the operation to be output to the display, the graphical output comprising a graphical representation based on the initial prediction.

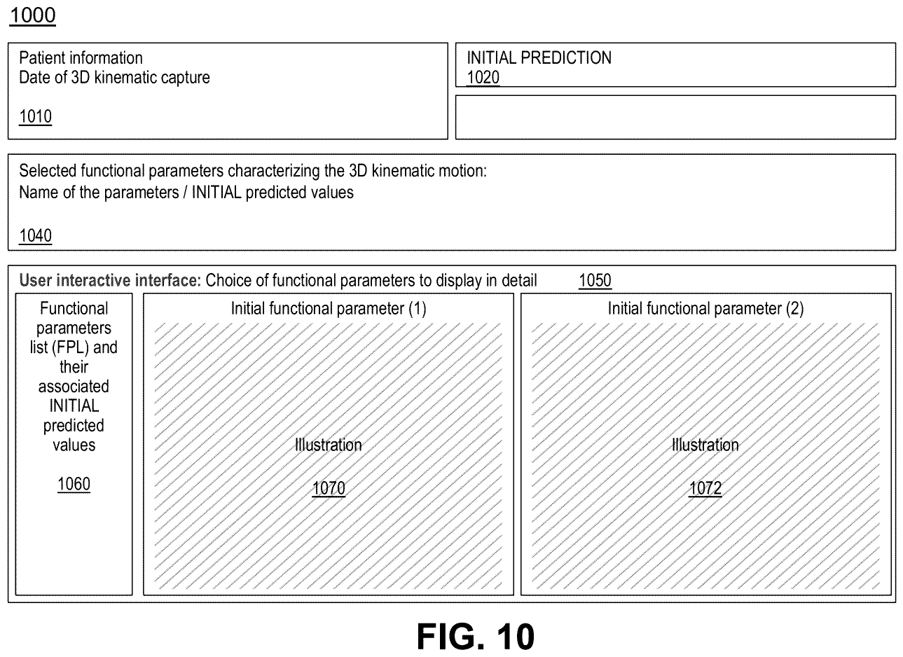

According to processing step 308, the processing device generates a representation—or visualization—of the initial prediction. This may include the representation of the initial prediction of the kinematic motion of the joint and representation of the impact of the surgical intervention on the functional parameters (pre-op) which can be used to plan the surgery and/or train for surgery. The visualization of the initial prediction may appear, for example, in the user interface 1000 as shown in FIG. 10.

For example, the user may use computer-assisted technologies (e.g., AR or VR headset, digital surgical system, hologram, holograph, etc.). The visualization may show the impact of the surgical intervention in real time pre-op (e.g., during a simulation) and/or intra-op (e.g., during a surgery). For example, in augmented reality (AR), a graphical guide may be displayed on one side of the field of vision to only partially overlay on top of what the surgeon sees. Examples of computer-assisted technologies include, but are not limited to, MAKO-Stryker, CORI-Smith&Nephew, Velys-Johnson&Jonhson DePuy-Synthes, NextAR-MedActa, ROSA-Zimmer Biomet, Orthopilot-Aesculap, GPS-Exactech, TMINI-ThinkSurgical, Knee+AR-PixeeMedical, and Arvis-Enovis.

For an example of a representation of the initial prediction, the representation may include different sections, such as a global functional score, shortened list (e.g., three items) of what the surgeon should pay attention to, the functional WB task, biomechanical markers, indicators, functional concepts, and/or functional scores.

The global functional score may be for a particular functional WB task such as gait. The score may be a numerical score, such as score out of 100 (e.g., 54 out of 100).

The shortened list of what the surgeon should pay attention to may include one or more functional parameters (e.g., biomechanical markers) of interest and an associated quantity/measurement, the affiliated kinematic phenotype for the patient, an evaluation of predisposing risk factors of pain after total knee arthroplasty (TKA), a classification type related to a functional concept, etc. For example, one of the biomechanical markers may be varus thrust at 3.6 degrees. Another biomechanical marker may be dynamic flexion contracture at 18 degrees. The kinematic phenotype may be a label such as ‘1’ (or ‘A’). The predisposing risk factor of pain after TKA may be labelled as one of low, moderate, or high (either discretely or on a continuum). The classification type related to a functional concept may be, for example, medial pivot. Another classification type related to a functional concept may be, for example, a varus-to-neutral type (or labelled as ‘type 12’) of dynamic coronal plane alignment of the knee from static alignment (or “dynamic CPAK”).

The functional WB task may be walking, sitting down, standing up, going up and down stairs or a stepper unit, squatting, lunging, leg pressing, hopping, jumping, or running. Alternatively, the functional WB task may be shown in categories, such as gait.

The biomechanical markers may include functional hip-knee-ankle angle (fHKA) (e.g., 7.1 degrees), varus heel strike angle (e.g., 9.2 degrees), varus minimum angle (e.g., 3.7, degrees), mean varus-valgus angle (e.g., 2.7 degrees), varus thrust angle (e.g., 3.6 degrees), expected varus, and kinematic phenotype (e.g., ‘1’).

The indicators may include frontal plane (by phenotype), frontal plane dynamic alignment, etc. For example, for the frontal plane indicator, there may be an associated graph of frontal plane behavior during gait for phenotype 1, showing values between 0 degrees and 15 degrees (e.g., mostly between 5 degrees and 10 degrees). Also for example, for the dynamic alignment indicator, there may be an associated (one-dimensional) graph of frontal plane dynamic alignment, showing values between −5 degrees and 15 degrees.

The functional concepts may include pivot pattern during stance (e.g., ‘lateral pivot’), multi-pivot pattern during gait (e.g., ‘yes’), type of dynamic varus-valgus alignment of the knee (DVAK) from static alignment (e.g., ‘type 23’). These concepts may be associated with illustrations (e.g., using representations of a neutrally aligned knee in static and of a valgus aligned knee in dynamic to illustrate a DVAK type 23).

The functional scores may include gait functional scores, such as loading (e.g., 55%), stiff knee (e.g., 75%), gait speed (e.g., 100%), frontal plane behavior (e.g., 56%), medial compartment stress (e.g., 25%), and lateral compartment stress (e.g., 78%). These scores may be displayed graphically (e.g., using a radial graph).

If the complementary surgical data is obtained intra-op (e.g., as described in processing step 318), the initial prediction algorithm may be run with a new set of complementary data including intra-op data as inputs so that a new initial prediction is calculated. The corresponding refined visualizations can then be displayed in real time (or in a short period of time such as under 2 seconds) following adjustments of surgical features from this new reference.

According to processing step 310, the processing device runs an iterative algorithm, which may follow the visualization of the initial prediction (e.g., in real time or in a short period of time such as under 2 seconds). The iterative algorithm may be selected by the user as one option between the iterative algorithm and the boundaries algorithm. The iterative algorithm generates a refined prediction. At a high level, the iterative algorithm receives a set of surgical features and a prediction as inputs and generates a refined prediction integrating the impact on the prediction of this specific set of surgical features as output. The processing device may require a minimum amount of data for specific categories, for example, a minimum of two surgical features (e.g., implant type, implant positioning).

In at least one implementation, the processing device generates a refined prediction of the 3D kinematic motion based on the initial prediction and a selection of one or more surgical features that relate to the operation.

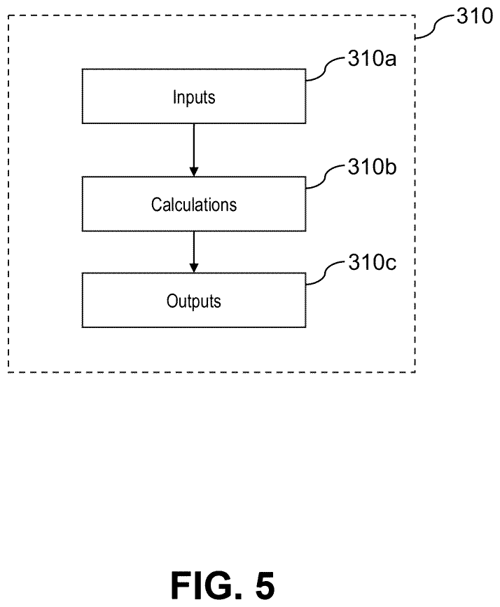

In at least one implementation, the processing step 310 runs the iterative algorithm shown in FIG. 5, using processing steps 310a, 310b, and 310c.

According to processing step 310a, the iterative algorithm takes as input a prediction (e.g., the initial prediction) and selected surgical features with specific values.

According to processing step 310b, the iterative algorithm performs calculations using the input from processing step 310a. These calculations may include one or more of: (1) processing all data inputs; and (2) rule-based calculation of functional parameters affected by the selected values for the surgical features.

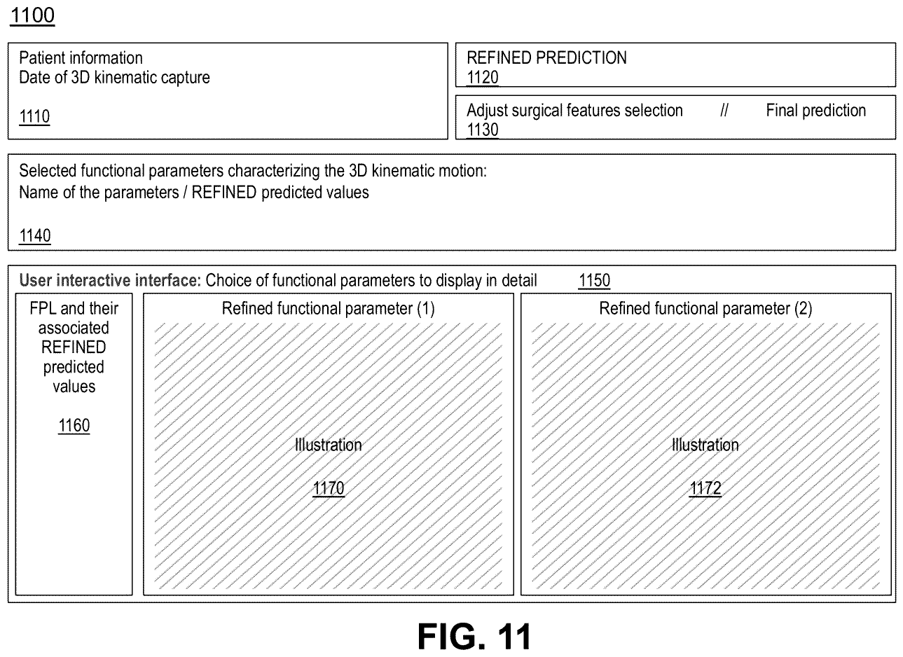

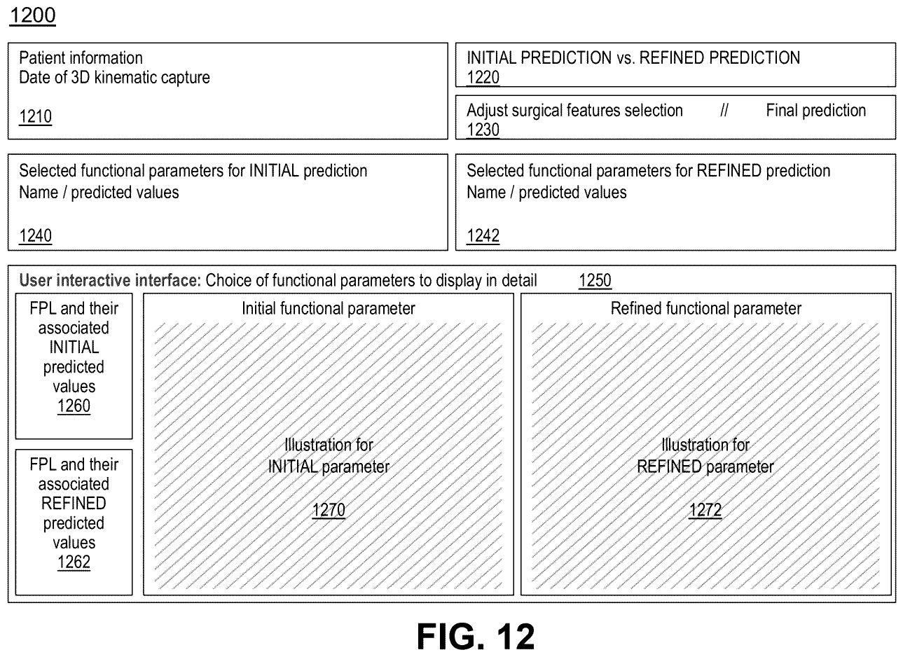

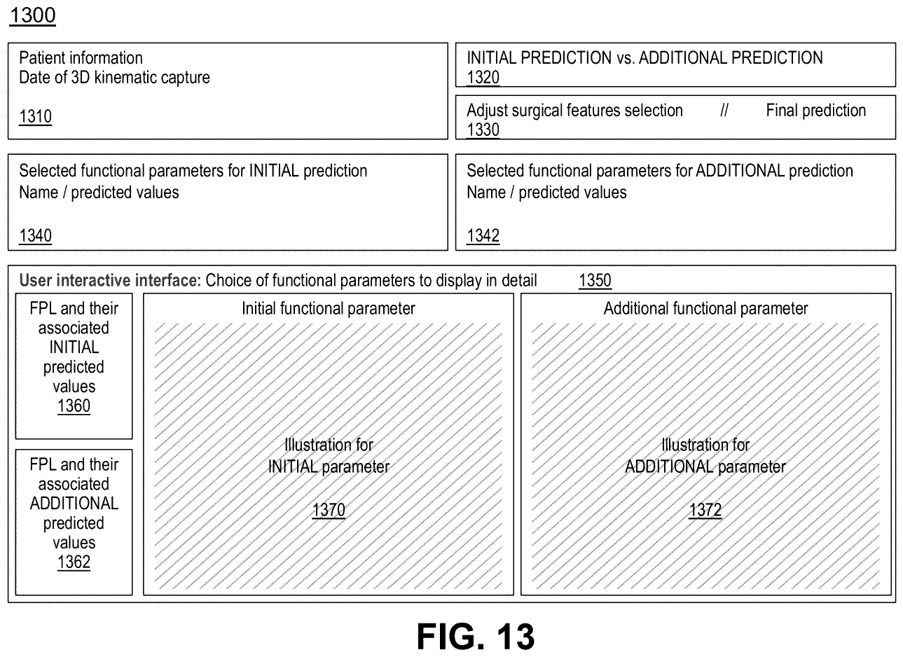

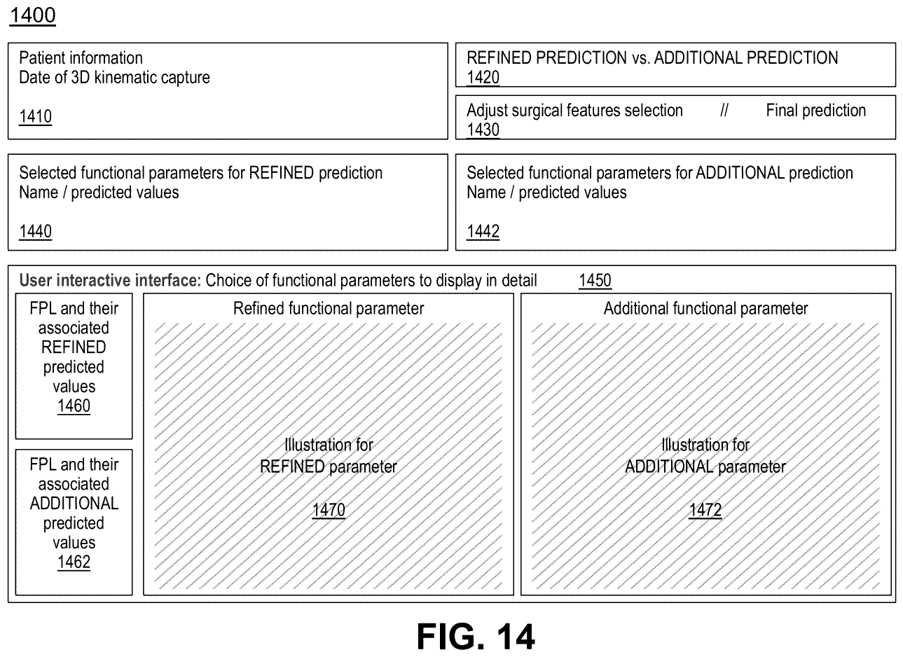

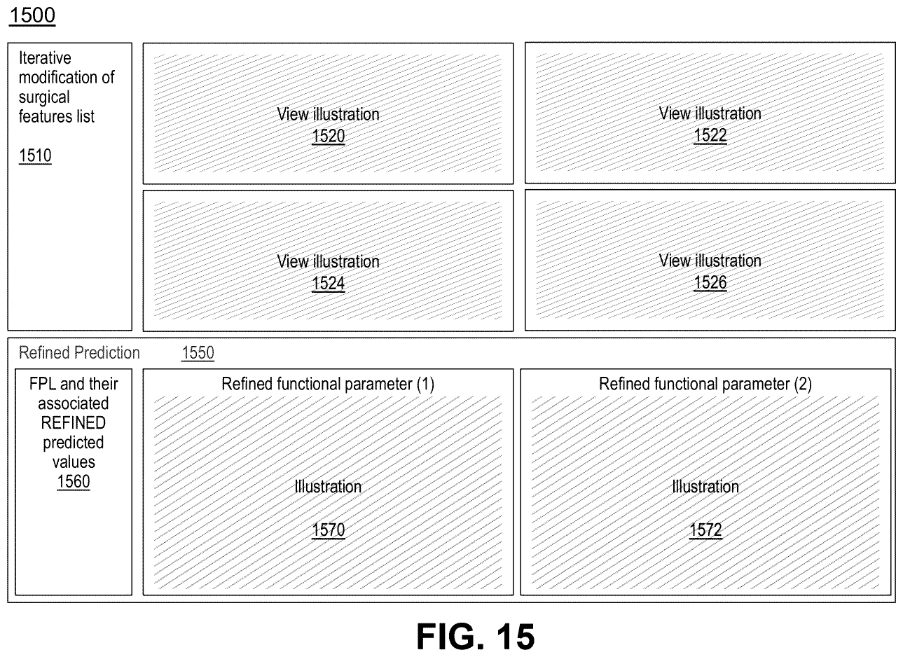

According to processing step 310c, the iterative algorithm generates a display of the refined prediction based on the calculations performed in processing step 310b. This display may include one or more of: (1) all calculated functional parameters modified with the impact of the set of selected surgical features; and (2) a summary of the selected surgical features with specific values used as input. The visualization of the refined prediction may appear, for example, in the user interface 1100 as shown in FIG. 11, the user interface 1200 as shown in FIG. 12, the user interface 1300 as shown in FIG. 13, the user interface 1400 as shown in FIG. 14, or the user interface 1500 as shown in FIG. 15.

According to processing step 312, the processing device runs a boundaries algorithm, which may follow the visualization of the initial prediction (e.g., in real time or in a short period of time such as under 2 seconds). The boundaries algorithm may be selected by the user as one option between the iterative algorithm and the boundaries algorithm. The boundaries algorithm generates a refined prediction. At a high level, the boundaries algorithm receives a set of functional parameters and their related boundaries (e.g., after a simulated surgery) and generates a range of surgical features to aim for in order to achieve a refined prediction (e.g., for post-op kinematic motion) within the set boundaries (or desired boundaries). The processing device may require a minimum amount of data for specific categories, for example, a minimum of two surgical features (e.g., implant type, implant positioning).

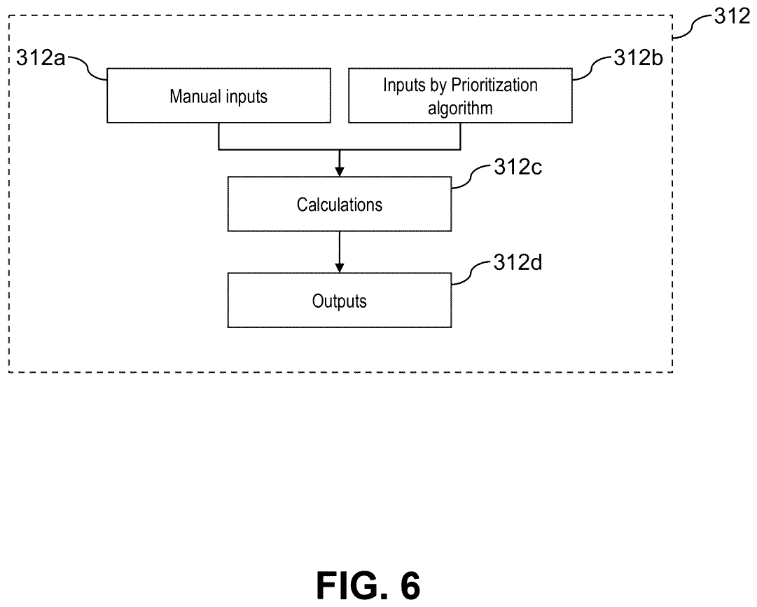

In at least one implementation, the processing step 312 runs the boundaries algorithm shown in FIG. 6, using processing steps 312a, 312b, 312c, and 312d.

According to processing step 312a, the boundaries algorithm takes as input “manual” inputs. The manual inputs may include selected functional parameters and/or desirable boundaries for these parameters in the refined prediction. Processing step 312a may be used instead of or in addition to processing step 312b.

According to processing step 312b, the boundaries algorithm takes as input specific recommendations from a prioritization algorithm. The recommendations may be based on kinematic phenotype affiliation, and include: (i) functional parameters to prioritize for optimal surgery and/or (ii) desirable related boundaries. Processing step 312b may be used instead of or in addition to processing step 312a.

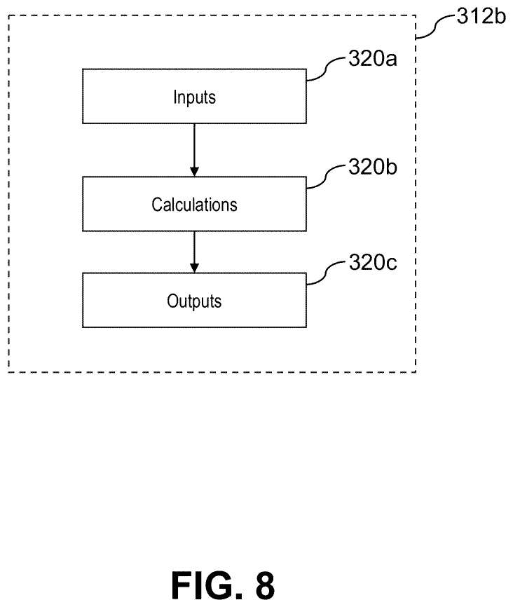

In at least one implementation, the processing step 312b runs the prioritization algorithm shown in FIG. 8, using processing steps 320a, 320b, and 320c.

According to processing step 320a, the prioritization algorithm takes as input a kinematic phenotype affiliation.

According to processing step 320b, the prioritization algorithm performs calculations using the input from processing step 320a. These calculations may include one or both of: (1) searching in a database which stores a small number (e.g., two, three, four) functional parameters to prioritize for each known phenotype for optimal surgery; and (2) desirable boundaries for these parameters stored in the same database.

According to processing step 320c, the prioritization algorithm generates outputs based on the calculations performed in processing step 320b. These outputs may include one or more of: (1) the small number of functional parameters to prioritize; and (2) related desirable boundaries for these parameters. These outputs may then be considered the specific recommendations for processing step 312b.

For an example of an application of the prioritization algorithm, suppose the user wants to obtain recommendations of the functional parameters to prioritize and their related boundaries to aim for optimal surgery based on the patient kinematic phenotype affiliation.

In this example, the prioritization algorithm is based on kinematic phenotype affiliation. There may be different kinematic phenotypes within knee surgery candidates, meaning that every patient from this population could be affiliated with one of these phenotypes. The first step may be to determine to which phenotype the initial prediction (3D kinematic data plus complementary surgical data) resembles the most. Alternatively, or in addition, the kinematic phenotype affiliation may be entered manually. This affiliation may be based on a nearest neighbor approach on these data inputs. Once the affiliation is done, the recommendations may be provided based on it. Each phenotype may have functional parameters which can be seen as assets or as downsides (kinematically speaking); the recommendations may focus on conserving the assets and correcting the downsides.

A particular kinematic phenotype [P] during gait may gather patients with a flexion contracture at heel strike (>10°, downside), a neutral knee dynamic alignment during stance ([−2°;2°], asset), and an internal rotation during loading ([−4°;−8°], asset).

Functional parameters may also be ranked between them based on their impact on clinical outcomes post-op in a specific phenotype, so the order of prioritization would be based on this ranking. For this phenotype [P], the most important parameter is the knee dynamic alignment during stance, followed by the flexion contracture at heel strike, and then the internal rotation during loading. The recommendations may focus on conserving the assets and correcting the downsides.

The recommendations for a patient from phenotype [P] may be: (i) keep neutral knee dynamic alignment at heel strike: [−2°;2°]; (ii) correct flexion contracture at heel strike: [2°;10°]; (iii) keep internal rotation during loading: [−4°;−8°]. In this example, these recommendations may address the fact that the knee dynamic alignment at heel strike has more impact on clinical outcomes than the flexion contracture at heel strike, and the internal rotation during loading.

According to processing step 312c, the boundaries algorithm performs calculations using the input from processing step 312a and/or 312b. These calculations may include rules-based calculations of boundaries for selected surgical features which maximize the odds of achieving a refined prediction of functional parameters within the desirable boundaries.

According to processing step 312d, the boundaries algorithm generates a display of the refined prediction based on the calculations performed in processing step 312c. This display may include one or more of: (1) all calculated surgical features with their related boundaries to achieve functional parameters within desirable boundaries; and (2) a summary of the selected functional parameters and their boundaries.

For an example of an application of the boundaries algorithm, suppose the user starts with the following simulated surgical plan intra-op:
(A) PS implant, femoral implant component size 3, tibial implant component size 5, femoral resections 6.5-7.5 mm, tibial resections 5.5-5.5 mm.

If the 3D kinematic data used as inputs are obtained during walking, the processing device generates a list of predicted functional parameters describing the knee dynamic behavior in weight-bearing conditions post-op if this surgery (A) is indeed done. This list may contain, for example: (i) knee dynamic alignment at heel strike: 3 degrees to 4.5 degrees; (ii) flexion amplitude during loading: 5 degrees to 8 degrees; (iii) etc.

At this point, before starting to actually carry out with the surgery (A), the user/surgeon has the option to set boundaries for one or more of the predicted functional parameters from the list. In the example above, the user can indicate, for example: (i) desired knee dynamic alignment at heel strike: [4°;6°]; (ii) desired flexion amplitude during loading: [6°;10°]; (iii) etc.

The boundaries algorithm generates ranges for the surgical features to aim for in order to obtain the “desired functional parameters” within these boundaries. The range for the corresponding surgical features may be, for example: (i) femoral implant component size: 3; (ii) tibial implant component size: 5; (iii) femoral resections: [4.5 mm; 6 mm]-[6 mm; 7 mm]; (iv) tibial resections: [5 mm; 5.5 mm]-[5 mm; 5.5 mm].

According to processing step 314, the processing device generates a representation—or visualization—of the refined prediction. The processing device may return to processing step 310 or processing step 312 to generate an additional (or subsequent) prediction, which may then replace the previous refined prediction with a new refined prediction.

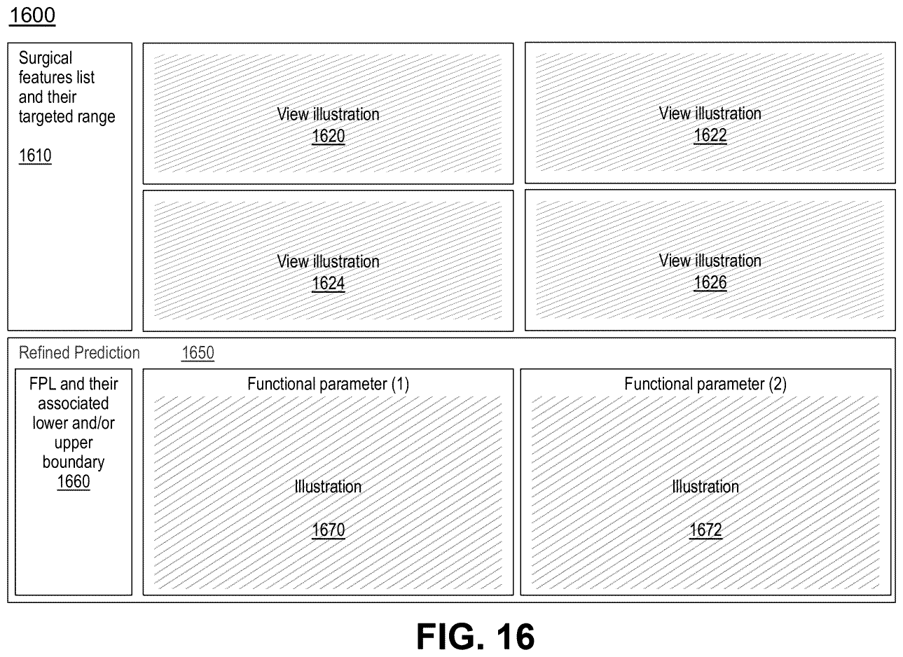

The visualization of the refined prediction may appear, for example, in the user interface 1100 as shown in FIG. 11 (e.g., when displaying just a refined prediction), the user interface 1200 as shown in FIG. 12 (e.g., when comparing an initial prediction to a refined prediction), the user interface 1300 as shown in FIG. 13 (e.g., when comparing an initial prediction to an additional or subsequent prediction), the user interface 1400 as shown in FIG. 14 (e.g., when comparing a refined prediction to an additional or subsequent prediction), the user interface 1500 as shown in FIG. 15 (e.g., when displaying iterative modifications of surgical features and associated refined or additional or subsequent predictions), or the user interface 1600 as shown in FIG. 16 (e.g., when displaying boundaries of functional parameters in a desired refined prediction and a targeted range for surgical features to achieve them). In at least one implementation, the processing device generates a graphical output (e.g., in real time) before or during the operation to be output to the display, the graphical output comprising a graphical representation based on the initial prediction and/or the refined prediction.

For example, the user may use computer-assisted technologies (e.g., AR or VR headset, digital surgical system, hologram, holograph, etc.). The visualization may show the impact of the surgical intervention in real time pre-op (e.g., during a simulation) and/or intra-op (e.g., during a surgery). For example, in augmented reality (AR), a graphical guide may be displayed on one side of the field of vision to only partially overlay on top of what the surgeon sees.

For a first example, for an application of the iterative algorithm, suppose the user uses the iterative algorithm and modifies the femoral component position from a valgus of 0 degrees to a varus of 3 degrees. The result may be a modification of the functional parameters prediction accordingly, for example, the varus thrust being reduced from 2.55 degrees to 0.50 degrees.

In this first example, the representation of the refined prediction may include different sections, such as a front view of the top part of the knee (e.g., femur), a front view of the bottom part of the knee (e.g., tibia), a top view of the top part of the knee (e.g., femur), a top view of the bottom part of the knee (e.g., tibia), a femoral implant component size, a tibial implant component size, and a bearing insert thickness and design, as well as selections for functional parameters (e.g., flexion loading angle, varus thrust angle, etc.), and a kinematic phenotype affiliation, which may show related information and/or graphs when selected.

The front view of the top part of the knee may indicate its alignment (e.g., component valgus), show an angle (e.g., 0 degrees), and show femoral resection measures on each compartment (e.g., 7 mm medial and 6 mm lateral). The front view of the bottom part of the knee may indicate its alignment (e.g., component valgus), show an angle (e.g., 0 degrees), and show tibial resection measures on each compartment (e.g., 12 mm medial and 8.5 mm lateral). The top view of the top part of the knee may show femoral posterior resection measures on each compartment (e.g., 9.5 mm medial and 12 mm lateral). The top view of the bottom part of the knee may show just the localization of the tibial implant.

The femoral implant component size may show a standardized size (e.g., size 5). The tibial implant component size may show a standardized size (e.g., size 4). The bearing insert thickness may show an actual thickness (e.g., 9 mm). This may stay the same after the first iteration, as the user may choose to keep this fixed.

The flexion loading angle may show as 4.2 degrees, then show as 5.5 degrees after the first iteration.

The phenotype may be shown as “1” and stay the same after the first iteration, because the first iteration would not modify the phenotype affiliation.

The varus thrust angle may show as 2.55 degrees, then show as 0.50 degrees after the first iteration. A graph may show where the angle lies within different ranges, such as good, bad, worst (e.g., 0 to 1.5 degrees is good, 1.5 to 2.5 degrees is bad, and 2.5 to 3.5 degrees is worst). In this first example, the varus thrust angle is reduced from 2.5 degrees (in the “worst” range) to 0.50 degrees (in the “good” range).

In this first example, the event that the position of the femoral component is moved with the first iteration (e.g., from 0 degrees to 3 degrees of varus), there may be a modification of the resections accordingly.

For a second example, for an application of the boundaries algorithm, suppose the user uses the boundaries algorithm and modifies functional parameters to determine which surgical features to aim for. The modification may be, for example, changing the varus thrust from a range of 3 degrees to 5 degrees to a range of 0 degrees to 2 degrees. The boundaries algorithm determines that the surgical features to aim for then are placing the femoral component between 3 degrees and 4 degrees of varus and the tibial component between 0 degrees and 0.5 degrees of valgus.

In this second example, the representation of the refined prediction may include different sections, such as a front view of the top part of the knee, a front view of the bottom part of the knee, a top view of the top part of the knee, a top view of the bottom part of the knee, a femoral component size, a tibial component size, and a bearing insert thickness, as well as functional parameters boundaries.

The front view of the top part of the knee may indicate its alignment and show an angle (e.g., 0 degrees), then show its alignment with a range of angles of 3 degrees to 4 degrees after modification; the femoral resection measures on each compartment may show as 7 mm and 6 mm, then show as 6 mm and 7.5 mm after modification. The front view of the bottom part of the knee may indicate its alignment and show an angle of 0 degrees, then show its alignment with a range of angles of 0 degrees to 0.5 degrees; the tibial resection measures on each compartment may show as 12 mm and 8.5 mm, then show as 10 mm and 6.5 mm after modification. The top view of the top part of the knee may show femoral posterior resection measures on each compartment as 9.5 mm and 12 mm, then show as 8 mm and 11.5 mm after modification. The top view of the bottom part of the knee may show just the localization of the tibial implant.

The femoral component size may show a standardized size (e.g., size 5). The tibia size may show a standardized size (e.g., size 4). The bearing insert thickness may show an actual thickness (e.g., 9 mm). This may stay the same after the first iteration, as the boundaries algorithm determines to keep this fixed.

The functional parameters boundaries may show varus thrust of 3 degrees to 5 degrees, then 0 degrees to 2 degrees after modification, and flexion during loading of 6 degrees to 10 degrees, then 6 degrees to 10 degrees (i.e., no change) after modification. In this second example, the boundaries algorithm shows how to modify the surgical features to aim for based on the user's modification of functional parameters. FIG. 16 shows an example user interface that provides a display for the boundaries algorithm.

According to processing step 316, the processing device runs a post-op (i.e., after the operation) algorithm. This may follow the visualization of the refined prediction (e.g., in real time or in a short period of time such as under 2 seconds). Alternatively, this may follow the visualization of the initial prediction (e.g., in real time or in a short period of time such as under 2 seconds).

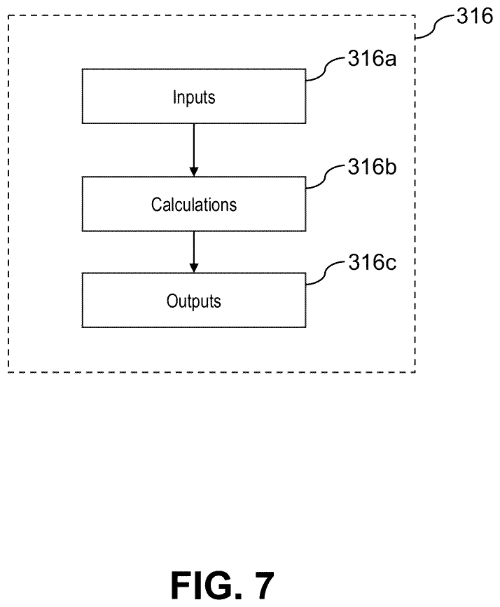

In at least one implementation, the processing step 316 runs the post-op algorithm shown in FIG. 7, using processing steps 316a, 316b, and 316c.

According to processing step 316a, the post-op algorithm takes as input a final prediction (e.g., all calculated functional parameters).

According to processing step 316b, the post-op algorithm performs calculations using the input from processing step 316a. These calculations may include one or more of: (1) rule-based prioritization of which small number of parameters to address with targeted post-op treatment recommendations, based on the value of each functional parameter used as input; (2) search in a database which stores desirable boundaries and treatment recommendations for each known functional parameter (and combination of functional parameters); and (3) treatment recommendations including at least tailored neuromuscular exercises and optionally other recommendations (e.g. bracing, etc.).

According to processing step 316c, the post-op algorithm generates a display of the patient-specific post-op recommendations based on the calculations performed in processing step 316b. This display may include one or more of: (1) the small number of functional parameters to target in post-op care with related desirable boundaries; (2) a small number of (e.g., two, three, four) neuromuscular exercises for each functional parameter to prioritize; and (3) a bracing recommendation (i.e., whether to have bracing and, if so, which type of brace).

At a high level, the post-op algorithm may generate patient-specific treatment recommendations for post-op care (i.e., post-op planning) based on the final kinematic motion prediction (once the optimal surgery is planned). These recommendations aim to target the final predicted functional parameters (e.g., where functional parameters are metrics that characterize joint function), in other words, possible residual deficiencies, and fully restore joint function.

For a first example of an application of the post-op algorithm, suppose the joint is the knee and the functional parameters values corresponding to a 100% functional knee are known. In a 100% functional knee, the frontal plane dynamic alignment at heel strike should be between −2 degrees and +2 degrees (i.e., “functional boundaries”), the flexion angle at heel strike should be between 0 degrees and 5 degrees, etc. So “fully restored” knee function means correcting/addressing/targeting each functional parameter until they are within their “functional boundaries”. These corrections can be obtained with a combination of tailored neuromuscular exercises combined (or not) with bracing.

In this first example, the final kinematic motion prediction may provide an estimation of what the functional parameters of the knee will look like post-op. The patient-specific treatment recommendations for post-op care may focus on correcting the functional parameters which are predicted to be outside of their “functional boundaries”.

The final kinematic motion prediction may be, for example: (i) frontal plane knee dynamic alignment at heel strike: [−2°;2°]; (ii) flexion angle at heel strike: [5°;8°]; (iii) etc. The treatment recommendations may include 3 exercises aiming to reduce the flexion angle at heel strike so it reaches [0°;5°]. These recommendations cannot be based on interim kinematic motion predictions because they can be far from what the functional parameters would look like post-op (i.e., if the user makes several adjustments in the surgical plan which affect the final functional parameters prediction).

The recommendations suggested to address functional parameters may include, for example, one or more of: (1) knee flexion contracture reduction; (2) knee flexion motion control during loading; (3) knee terminal extension correction at stance; (4) knee flexion correction at push-off; (5) knee flexion reduction at initial contact; (6) exaggerated knee extension control at heel strike; (7) exaggerated knee extension control during stance; (8) static varus alignment correction; (9) static valgus correction; (10) dynamic varus-valgus movement control; (11) varus thrust reduction; (12) dynamic valgus movement control; (13) valgus control at push-off; (14) valgus control during stance; (15) valgus control at initial contact; (16) external tibial rotation correction at heel strike; (17) external tibial rotation correction during push-off; (18) internal tibial rotation control during loading.

For a second example of an application of the post-op algorithm, suppose the functional parameter “flexion amplitude during loading” is identified as having a residual deficiency. If the value of this functional parameter is [0°;8°], that means that the patient has a flexion absorption deficiency—a patient with a fully functional knee would be able to flex it more than 8 degrees during the loading phase of gait.

In this second example, the post-op algorithm may generate a list of recommendations (e.g., with explanations and images to educate the patient on the importance of addressing the residual deficiency and to describe how to perform the exercises). These recommendations may include: (i) stretching exercise for the hamstring (2 sets of 30 seconds of holding per day); (ii) neuromuscular exercise focusing on knee bending at the beginning of each step while walking (2 sets of 10 repetitions per day); (iii) no bracing; (iv) etc.

According to processing step 318, the processing device receives data collected intra-op. This data may form the complementary surgical data. Alternatively, this data may add to the complementary surgical data. For example, the data can be input manually by the surgeon or an assistant (e.g., while the surgeon moves the knee through a passive movement, they indicate to an assistant that the balance gap during passive movement at 0 degrees is 2.5 mm, at 10 degrees is 2 mm, at 20 degrees is 1.5 mm, etc.). Also, for example, the data can be collected with a computer-assisted surgical technology (e.g., the surgical robot registers the balance gap while the surgeon moves the knee during passive movement).

The method 300 then ends.

In at least one implementation of the method 300, the 3D kinematic motion data is obtained before the operation.

In at least one implementation of the method 300, the 3D kinematic motion data comprises at least a position and a time.

In at least one implementation of the method 300, the 3D kinematic motion data relates to a bone involved in the joint, irrespective of any physical force applied to the bone.

In at least one implementation of the method 300, the 3D kinematic motion data relates to a bone involved in the joint, irrespective of any physical force applied to the joint.

In at least one implementation of the method 300, the capturing system comprises at least one of: a video camera, a stereo camera, a light detection and ranging (LiDAR) sensor, a gyroscopic sensor, an inertial measurement unit, an accelerometer sensor, a markerless motion capture system, an electromagnetic sensor, an optical motion sensor, a radiographic system, or a custom motion sensor configured to obtain the 3D kinematic motion data of the joint.

In at least one implementation of the method 300, the capturing system processes some or all of the 3D kinematic motion data that is captured.

In at least one implementation of the method 300, the 3D kinematic motion data is captured during one or more WB tasks in a close or open kinematic chain, the WB tasks comprising at least one of: walking, sitting down, standing up, stepping up a stair or module, stepping down the stair or module, kneeling, going into deep flexion, hopping, squatting, lunging, leg pressing, running, or jumping.

In at least one implementation of the method 300, the complementary surgical data comprises at least one of: two-dimensional (2D) imaging data, 3D imaging data, digital surgical system data, image-based surgical robotic-assisted system data, imageless surgical robotic-assisted system data, computer-aided surgical system data, surgical feature data, static joint alignment data, dynamic joint alignment data during passive movement, joint kinematic data during passive movement, joint kinematic data during non-weight-bearing active movement, soft tissue behavior data, physical data, clinical data, or socio-demographic data.

In at least one implementation of the method 300, the functional parameters comprise at least one of: a biomechanical marker, joint stability information, joint soft tissue behavior, ligamentous laxities, range of motion, a functional concept or classification, a functional score, a kinematic phenotype affiliation, a dynamic alignment behavior, a pattern difference between static and dynamic joint alignment, a pattern difference between parameters during weight-bearing vs. non-weight-bearing activities, or a pattern difference between parameters during passive vs. active movement for both weight-bearing and non-weight-bearing activities of the joint.

In at least one implementation of the method 300, the initial prediction comprises at least one of a 3D joint kinematic pattern prediction or the one or more functional parameters.

In at least one implementation of the method 300, the one or more surgical features comprise at least one of: an orthopedic implant type, an orthopedic implant size, a bearing insert design, a bearing insert size, orthopedic implant 3D positioning, resection measures, or soft-tissue balance.

In at least one implementation of the method 300, the one or more surgical features comprise the orthopedic implant type and the orthopedic implant 3D positioning.

In at least one implementation of the method 300, the refined prediction is generated by assessing the impact of the selection of the one or more surgical features on the 3D kinematic motion as compared to the initial prediction.

In at least one implementation of the method 300, the complementary surgical data comprises operation data collected in real time during the operation.

In at least one implementation of the method 300, the method 300 further comprises loading a recommendation for postoperative care from a database according to the refined prediction when the refined prediction is not followed by an additional refined prediction.

In at least one implementation of the method 300, the method 300 further comprises generating an additional refined prediction based on an impact of a modification of the selection of the one or more surgical features on the 3D kinematic motion as compared to the refined prediction.

In at least one implementation of the method 300, the graphical output comprises an updated graphical representation of the additional refined prediction next to the initial prediction.

In at least one implementation of the method 300, the method 300 further comprises generating subsequent refined predictions based on an impact of subsequent modifications of the selection of the one or more surgical features on the 3D kinematic motion as compared to the refined prediction.

In at least one implementation of the method 300, the graphical output comprises a subsequent updated graphical representation of the subsequent refined prediction next to the initial prediction or the refined prediction.

In at least one implementation of the method 300, the display comprises at least one of: a monitor, a smartphone, a computer, a laptop, a tablet, a virtual reality headset, an augmented reality headset, a holographic display, a spatial apparatus, or a custom apparatus used to display visual information and/or data.

In at least one implementation of the method 300, the graphical output is configured to display the graphical representation of the initial prediction alone, the refined prediction alone, or a combination of the initial prediction and the refined prediction.

In at least one implementation of the method 300, the graphical output is configured to display the graphical representation of the additional refined prediction alone, or a combination of the initial prediction and the additional refined prediction.

In at least one implementation of the method 300, the graphical output is configured to display the graphical representation of the subsequent refined prediction alone, or a combination of the initial prediction and the subsequent refined prediction.

In at least one implementation of the method 300, the method 300 further comprises: receiving at least one of a lower boundary or an upper boundary on the one or more functional parameters associated with at least one of the initial prediction or the refined prediction; and calculating a targeted range for the selection of the one or more surgical features to maximize an expected outcome of achieving the 3D kinematic motion exhibiting the one or more functional parameters within the at least one of the lower boundary or the upper boundary after the operation. The graphical output may comprise a graphical representation of the at least one of the lower boundary or the upper boundary and the targeted range.

In at least one implementation of the method 300, the one or more functional parameters is a kinematic phenotype affiliation and the at least one of the lower boundary or the upper boundary is loaded from a database.

In at least one implementation, the method 300 is performed in a different order. In at least one implementation, the method 300 is performed in such a manner that some or all of the steps are carried out iteratively.

In at least one embodiment, a first modified method for generating a prediction of 3D kinematic motion of a joint before or during an operation comprises only some of the steps in method 300. In such a case, the first modified method may replace some of the missing steps with other steps to further achieve the goals of the present technology.

In at least one embodiment, a second modified method for generating a prediction of 3D kinematic motion of a joint before or during an operation comprises all of the steps in method 300, as well as one or more additional steps. In such a case, the second modified method may add steps to further achieve the goals of the present technology.

In at least one embodiment, a non-transitory computer-readable medium (CRM) has stored thereon computer-readable instructions that, when executed by at least one processor, cause the at least one processor to perform the method 300. The CRM may also have stored thereon alternative, additional, or fewer computer-readable instructions, such as those of any of the implementations of method 300 discussed herein.

In at least one embodiment, a system for generating a prediction of 3D kinematic motion of a joint before or during an operation according to method 300 comprises a display and at least one processor. The system may also comprise a capturing system that is configured to capture the 3D kinematic motion of the joint. The display is configured to display a representation of the prediction of the 3D kinematic motion of the joint. The at least one processor is operatively connected to the display and a non-transitory storage medium having stored thereon computer-readable instructions. The at least one processor may also be operatively connected to the capturing system. The computer-readable instructions, when executed by the at least one processor, cause the at least one processor to perform the method 300. The non-transitory storage medium may also have stored thereon alternative, additional, or fewer computer-readable instructions, such as those of any of the implementations of method 300 discussed herein.

### Method for Generating and Displaying 3D Kinematic Motion of a Joint During Weight-Bearing Activity Before or During an Operation

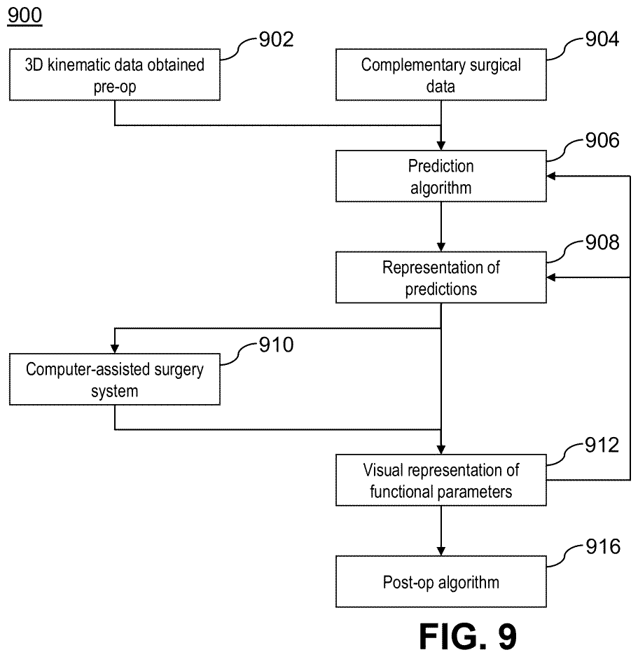

FIG. 9 depicts a flowchart of a method 900 for generating and displaying 3D kinematic motion of a joint during weight-bearing activity before or during an operation in accordance with one or more non-limiting implementations of the present technology. The method 900 may be used for generating and displaying 3D kinematic motion of a human joint during weight-bearing activity (before and/or during surgery) as well as the predicted impact of a surgical intervention on this kinematic motion.

The method 900 may be considered to be a modified version of method 300, where similar processing steps of method 900 may incorporate none, some, or all of the features of corresponding processing steps of method 300. For example, processing step 902 may incorporate some features of processing step 302, processing step 904 may incorporate some features of processing step 304, processing step 906 may incorporate some features of processing step 306, processing step 908 may incorporate some features of processing step 308, and processing step 916 may incorporate some features of processing step 316.

In one or more implementations, the server 220 comprises a processing device such as the processor 110 and/or the GPU 111 operatively connected to a non-transitory computer readable storage medium such as the solid-state drive 120 and/or the random-access memory 130 storing computer-readable instructions. The processing device, upon executing the computer-readable instructions, is configured to or operable to execute the method 900.

The method 900 begins at one or both of processing steps 902 or 904. For example, the method 900 may begin with processing step 902 then continue with another processing step. Also, for example, the method 900 may begin with processing step 902 then continue with processing step 904, before proceeding to another processing step. Also, for example, the method 900 may begin with processing steps 902 and 904 being executed in combination, independently, or in parallel.

According to processing step 902, the processing device receives 3D kinematic data obtained pre-op (i.e., before an operation). The 3D kinematic data may be received, for example, from a “capturing system”. In at least one implementation, the 3D kinematic data obtained pre-op may be 3D motion estimations (e.g., visual estimations, modelled estimations, etc.) instead of (or in addition to) 3D kinematic data collected from a capturing system.

According to processing step 904, the processing device receives complementary surgical data. The complementary surgical data may be received from a capturing system or from user input related to the planned surgery.

According to processing step 906, the processing device runs a prediction algorithm using the 3D kinematic data and the complementary surgical data. In at least one implementation, the processing device may run the prediction algorithm using the 3D kinematic data without complementary surgical data. The prediction algorithm may be an initial prediction algorithm or a refined prediction algorithm.

According to processing step 908, the processing device generates a representation—or visualization—of the predictions.

According to processing step 910, the processing device provides an option to use a computer-assisted surgery system. For example, the surgeon can choose to conduct the surgery with or without the computer-assisted surgery system.

According to processing step 912, the processing device displays a representation—or visualization—of the functional parameters. For example, the user may use computer-assisted technologies (e.g., AR or VR headset, digital surgical system, hologram, holograph, etc.) and visualize the predictions of the functional parameters and the impact of the surgical intervention in real time pre-op (e.g., during a simulation) and/or intra-op (e.g., during a surgery). For example, in augmented reality (AR), a graphical guide may be displayed on one side of the field of vision to only partially overlay on top of what the surgeon sees. The method 900 may return to processing step 906 or processing step 908, for example, for prediction refinements.

According to processing step 916, the processing device runs a post-op (i.e., after the operation) algorithm. For example, once the final optimal surgery is selected, the user can generate patient-specific treatment recommendations for post-op care management based on the final kinematic motion prediction.

In at least one implementation, the method 900 is performed in a different order. In at least one implementation, the method 900 is performed in such a manner that some or all of the steps are carried out iteratively.

In at least one embodiment, a first modified method for generating and displaying 3D kinematic motion of a joint during weight-bearing activity before or during an operation comprises only some of the steps in method 900. In such a case, the first modified method may replace some of the missing steps with other steps to further achieve the goals of the present technology.

In at least one embodiment, a second modified method for generating and displaying 3D kinematic motion of a joint during weight-bearing activity before or during an operation comprises all of the steps in method 900, as well as one or more additional steps. In such a case, the second modified method may add steps to further achieve the goals of the present technology.

### Visualizations for Predictions of 3D Kinematic Motion of a Joint Before or During an Operation

FIGS. 10 to 16 depict schematic diagrams for example of user interfaces that provide representations—or visualizations—for predictions of 3D kinematic motion of a joint before or during an operation. One or more of these visualizations may be used in one or more parts of method 300 or method 900. The visualizations can be for an initial prediction (e.g., a 1st prediction), a refined prediction (e.g., a 2nd prediction), an additional prediction (e.g., a 3rd prediction), or subsequent predictions (e.g., 4th, 5th, 6th predictions).

FIG. 10 depicts a schematic diagram of a user interface 1000 for an initial prediction of 3D kinematic motion of a joint. Section 1010 shows patient information and a date of 3D kinematic capture. Section 1020 indicates that the visualization is for an initial prediction. Section 1040 shows selected functional parameters characterizing the 3D kinematic motion; the names of the parameters; and initial predicted values. Section 1050 shows a user interactive interface, with a choice of functional parameters to display in detail. Section 1060 shows a functional parameters list (FPL) and their associated initial predicted values. Section 1070 shows an illustration of a first initial functional parameter, the illustration facilitating the understanding for the user (e.g., radars, curves, gradual lines, etc.). Section 1072 shows an illustration of a second initial functional parameter, the illustration facilitating the understanding for the user (e.g., radars, curves, gradual lines, etc.).

FIG. 11 depicts a schematic diagram of a user interface 1100 for a refined prediction of 3D kinematic motion of a joint. Section 1110 shows patient information and a date of 3D kinematic capture. Section 1120 indicates that the visualization is for a refined prediction. Section 1130 indicates that the user interface provides the user an adjustment of surgical features selection or that it is a final prediction. Section 1140 shows selected functional parameters characterizing the 3D kinematic motion; the names of the parameters; and refined predicted values. Section 1150 shows a user interactive interface, with a choice of functional parameters to display in detail. Section 1160 shows a functional parameters list (FPL) and their associated refined predicted values. Section 1170 shows an illustration of a first refined functional parameter, the illustration facilitating the understanding for the user (e.g., radars, curves, gradual lines, etc.). Section 1172 shows an illustration of a second refined functional parameter, the illustration facilitating the understanding for the user (e.g., radars, curves, gradual lines, etc.).

FIG. 12 depicts a schematic diagram of a user interface 1200 for a comparison of an initial prediction with a refined prediction of 3D kinematic motion of a joint. Section 1210 shows patient information and a date of 3D kinematic capture. Section 1220 indicates that the visualization is for a comparison between an initial prediction and a refined prediction. Section 1230 indicates that the user interface provides the user an adjustment of surgical features selection or that it is a final prediction. Section 1240 shows selected functional parameters (characterizing the 3D kinematic motion) for an initial prediction; the names of the parameters; and predicted values. Section 1242 shows selected functional parameters (characterizing the 3D kinematic motion) for a refined prediction; the names of the parameters; and predicted values. Section 1250 shows a user interactive interface, with a choice of functional parameters to display in detail. Section 1260 shows a functional parameters list (FPL) and their associated initial predicted values. Section 1262 shows a functional parameters list (FPL) and their associated refined predicted values. Section 1270 shows an illustration of an initial functional parameter, the illustration facilitating the understanding for the user (e.g., radars, curves, gradual lines, etc.). Section 1272 shows an illustration of a refined functional parameter, the illustration facilitating the understanding for the user (e.g., radars, curves, gradual lines, etc.).

FIG. 13 depicts a schematic diagram of a user interface 1300 for a comparison of an initial prediction with an additional (or subsequent) prediction of 3D kinematic motion of a joint. Section 1310 shows patient information and a date of 3D kinematic capture. Section 1320 indicates that the visualization is for a comparison between an initial prediction and an additional (or subsequent) prediction. Section 1330 indicates that the user interface provides the user an adjustment of surgical features selection or that it is a final prediction. Section 1340 shows selected functional parameters (characterizing the 3D kinematic motion) for an initial prediction; the names of the parameters; and predicted values. Section 1342 shows selected functional parameters (characterizing the 3D kinematic motion) for an additional (or subsequent) prediction; the names of the parameters; and predicted values. Section 1350 shows a user interactive interface, with a choice of functional parameters to display in detail. Section 1360 shows a functional parameters list (FPL) and their associated initial predicted values. Section 1362 shows a functional parameters list (FPL) and their associated additional (or subsequent) predicted values. Section 1370 shows an illustration of an initial functional parameter, the illustration facilitating the understanding for the user (e.g., radars, curves, gradual lines, etc.). Section 1372 shows an illustration of an additional (or subsequent) functional parameter, the illustration facilitating the understanding for the user (e.g., radars, curves, gradual lines, etc.).

FIG. 14 depicts a schematic diagram of a user interface 1400 for a comparison of a refined prediction with an additional (or subsequent) prediction of 3D kinematic motion of a joint. Section 1410 shows patient information and a date of 3D kinematic capture. Section 1420 indicates that the visualization is for a comparison between a refined prediction and an additional (or subsequent) prediction. Subsequent prediction may refer to the fact that it is subsequent to a refined prediction; alternatively, or in addition, subsequent prediction may refer to the fact that it is subsequent to a previous additional prediction. Section 1430 indicates that the user interface provides the user an adjustment of surgical features selection or that it is a final prediction. Section 1440 shows selected functional parameters (characterizing the 3D kinematic motion) for a refined prediction; the names of the parameters; and predicted values. Section 1442 shows selected functional parameters (characterizing the 3D kinematic motion) for an additional (or subsequent) prediction; the names of the parameters; and predicted values. Section 1450 shows a user interactive interface, with a choice of functional parameters to display in detail. Section 1460 shows a functional parameters list (FPL) and their associated refined predicted values. Section 1462 shows a functional parameters list (FPL) and their associated additional (or subsequent) predicted values. Section 1470 shows an illustration of a refined functional parameter, the illustration facilitating the understanding for the user (e.g., radars, curves, gradual lines, etc.). Section 1472 shows an illustration of an additional (or subsequent) functional parameter, the illustration facilitating the understanding for the user (e.g., radars, curves, gradual lines, etc.).

FIG. 15 depicts a schematic diagram of a user interface 1500 for iterative modification of surgical features of 3D kinematic motion of a joint. Section 1510 shows an iterative modification of surgical features list and their associated values. Section 1520 shows an illustration of a surgical feature in a first view (e.g., one femur view). Section 1522 shows an illustration of a surgical feature in a second view (e.g., a second femur view). Section 1524 shows an illustration of a surgical feature in a third view (e.g., one tibia view). Section 1526 shows an illustration of a surgical feature in a fourth view (e.g., a second tibia view). Section 1550 shows a refined prediction or an additional (or subsequent) prediction window. Section 1560 shows a functional parameters list (FPL) and their associated refined or additional (or subsequent) predicted values. Section 1570 shows an illustration of a refined functional parameter or a first additional (or subsequent) functional parameter, the illustration facilitating the understanding for the user (e.g., radars, curves, gradual lines, etc.). Section 1572 shows an illustration of the refined functional parameter (e.g., in a second view), the first additional (or subsequent) functional parameter (e.g., in a second view), or a second additional (or subsequent) functional parameter, the illustration facilitating the understanding for the user (e.g., radars, curves, gradual lines, etc.).

FIG. 16 depicts a schematic diagram of a user interface 1600 for boundaries of functional parameters of 3D kinematic motion of a joint. Section 1610 shows a surgical features list and their targeted range(s). Section 1620 shows an illustration of a surgical feature in a first view (e.g., one femur view). Section 1622 shows an illustration of a surgical feature in a second view (e.g., a second femur view). Section 1624 shows an illustration of a surgical feature in a third view (e.g., one tibia view). Section 1626 shows an illustration of a surgical feature in a fourth view (e.g., a second tibia view). Section 1650 shows a refined prediction or an additional (or subsequent) prediction window. Section 1660 shows a functional parameters list (FPL) and their associated lower and/or upper boundary. Section 1670 shows an illustration of a first functional parameter, the illustration facilitating the understanding for the user (e.g., radars, curves, gradual lines, etc.). Section 1672 shows an illustration of a second functional parameter, the illustration facilitating the understanding for the user (e.g., radars, curves, gradual lines, etc.).

It should be apparent to those skilled in the art that at least some implementations of the present technology aim to expand a range of technical solutions for addressing one or more particular technical problems faced by generating a prediction of 3D kinematic motion of a joint before or during an operation, such as inefficiency and cost (e.g., from a computing power standpoint) when operating on data in real time or on a large quantity of data, which may save computational resources, reduce computing power, or make more efficient use of network bandwidth.

It should be expressly understood that not all technical effects mentioned herein need to be enjoyed in each and every implementation of the present technology. For example, implementations of the present technology may be implemented without the user enjoying some of these technical effects, while other non-limiting implementations may be implemented with the user enjoying other technical effects or none at all.

Some of these steps and signal sending-receiving are well known in the art and, as such, have been omitted in certain portions of this description for the sake of simplicity. The signals can be sent-received using optical means (such as a fiber-optic connection), electronic means (such as using wired or wireless connection), and mechanical means (such as pressure-based, temperature based or any other suitable physical parameter based).

Modifications and improvements to the above-described implementations of the present technology may become apparent to those skilled in the art. The foregoing description is intended to be exemplary rather than limiting.

1. A system for generating a prediction of an impact of an intervention on three-dimensional (3D) kinematic motion of a joint before or during an operation, the system comprising:
a display configured to display a representation of the prediction of the impact of the intervention on the 3D kinematic motion of the joint; and
at least one processor operatively connected to the display and a non-transitory storage medium having stored thereon computer-readable instructions that, when executed by the at least one processor, cause the at least one processor to:
receive 3D kinematic motion data of the joint, the 3D kinematic motion data relating to the 3D kinematic motion during at least one of a weight-bearing action or the weight-bearing action in combination with motion during a non-weight-bearing action;
receive complementary surgical data that relate to the joint or the operation;
merge the 3D kinematic motion data and the complementary surgical data, thereby creating a merged data set;
derive, from the merged data set, one or more functional parameters characterizing the 3D kinematic motion;
determine an initial prediction of the 3D kinematic motion before or during the operation based on the one or more functional parameters;
generate a refined prediction of the 3D kinematic motion based on the initial prediction and a selection of one or more surgical features that relate to the operation; and
generate a graphical output before or during the operation to be output to the display, the graphical output comprising a graphical representation based on at least one of the initial prediction or the refined prediction.

2. The system of claim 1, wherein the 3D kinematic motion data is obtained from a capturing system that is configured to capture the 3D kinematic motion data.

3. The system of claim 1, wherein the 3D kinematic motion data comprises at least a position and a time.

4. The system of claim 2, wherein the system further comprises the capturing system and the capturing system comprises at least one of: a video camera, a stereo camera, a light detection and ranging (LiDAR) sensor, a gyroscopic sensor, an inertial measurement unit, an accelerometer sensor, a markerless motion capture system, an electromagnetic sensor, an optical motion sensor, a radiographic system, or a custom motion sensor configured to obtain the 3D kinematic motion data of the joint.

5. The system of claim 4, wherein the capturing system processes some or all of the 3D kinematic motion data that is captured.

6. The system of claim 1, wherein the initial prediction comprises at least one of a 3D joint kinematic pattern prediction or the one or more functional parameters.

7. The system of claim 1, wherein the refined prediction is generated by assessing the impact of the selection of the one or more surgical features on the 3D kinematic motion as compared to the initial prediction.

8. The system of claim 1, wherein the complementary surgical data comprises operation data collected in real time during the operation.

9. The system of claim 1, wherein the computer-readable instructions, when executed, further cause the at least one processor to load a recommendation for postoperative care from a database according to the refined prediction when the refined prediction is not followed by an additional refined prediction.

10. The system of claim 1, wherein the computer-readable instructions, when executed, further cause the at least one processor to generate an additional refined prediction based on an impact of a modification of the selection of the one or more surgical features on the 3D kinematic motion as compared to the refined prediction.

11. The system of claim 10, wherein the graphical output comprises an updated graphical representation of the additional refined prediction next to the initial prediction.

12. The system of claim 10, wherein the computer-readable instructions, when executed, further cause the at least one processor to generate subsequent refined predictions based on an impact of subsequent modifications of the selection of the one or more surgical features on the 3D kinematic motion as compared to the refined prediction.

13. The system of claim 12, wherein the graphical output comprises a subsequent updated graphical representation of the subsequent refined prediction next to the initial prediction or the refined prediction.

14. The system of claim 1, wherein the graphical output is configured to display the graphical representation of the initial prediction alone, the refined prediction alone, or a combination of the initial prediction and the refined prediction.

15. The system of claim 10, wherein the graphical output is configured to display the graphical representation of the additional refined prediction alone, or a combination of the initial prediction and the additional refined prediction.

16. The system of claim 13, wherein the graphical output is configured to display the graphical representation of the subsequent refined prediction alone, or a combination of the initial prediction and the subsequent refined prediction.

17. The system of claim 1, wherein the computer-readable instructions, when executed, further cause the at least one processor to:
receive at least one of a lower boundary or an upper boundary on the one or more functional parameters associated with at least one of the initial prediction or the refined prediction; and
calculate a targeted range for the selection of the one or more surgical features to maximize an expected outcome of achieving the 3D kinematic motion exhibiting the one or more functional parameters within the at least one of the lower boundary or the upper boundary after the operation;
wherein the graphical output comprises a graphical representation of the at least one of the lower boundary or the upper boundary and the targeted range.

18. The system of claim 17, wherein the one or more functional parameters is a kinematic phenotype affiliation and the at least one of the lower boundary or the upper boundary is loaded from a database.

19. A method for generating a prediction of an impact of an intervention on three-dimensional (3D) kinematic motion of a joint before or during an operation, the method comprising:
receiving 3D kinematic motion data of the joint, the 3D kinematic motion data relating to the 3D kinematic motion during at least one of a weight-bearing action or the weight-bearing action in combination with motion during a non-weight-bearing action;
receiving complementary surgical data that relate to the joint or the operation;
merging the 3D kinematic motion data and the complementary surgical data, thereby creating a merged data set;
deriving, from the merged data set, one or more functional parameters characterizing the 3D kinematic motion;
determining an initial prediction of the 3D kinematic motion before or during the operation based on the one or more functional parameters;
generating a refined prediction of the 3D kinematic motion based on the initial prediction and a selection of one or more surgical features that relate to the operation; and
generating a graphical output before or during the operation to be output to a display, the graphical output comprising a graphical representation based on at least one of the initial prediction or the refined prediction.

20. A non-transitory computer-readable medium having store thereon computer-readable instructions that, when executed by at least one processor, cause the at least one processor to perform the method according to claim 19.
